(19) United States
(12) Patent Application Publication
(10) Pub. No.: US 2026/0226056 A1
(43) Pub. Date: Aug. 6, 2026

(54) SUBSTITUTED 1H-PYRAZOLE-4-CARBOXAMIDES AS SARM1 INHIBITORS

(71) Applicant: DISARM THERAPEUTICS, INC., Indianapolis, IN (US)

(72) Inventors: Andrew Smith BREARLEY, Indianapolis, IN (US); Rajesh DEVRAJ, Indianapolis, IN (US); Robert Owen HUGHES, Indianapolis, IN (US); Shelley Anne PARROTT, Indianapolis, IN (US); Todd BOSANAC, Indianapolis, IN (US)

(21) Appl. No.: 19/150,442

(22) PCT Filed: Jan. 18, 2024
(86) PCT No.: PCT/US2024/011943

§ 371 Date: Jul. 23, 2025

Related U.S. Application Data
(60) Provisional application No. 63/481,202, filed on Jan. 24, 2023.

(51) Int. Cl. C07D 471/14; A61K 31/4375; A61K 31/4439; A61K 31/444; A61K 31/501; A61K 31/519; C07D 401/04; C07D 401/14; C07D 403/04; C07D 409/14; C07D 487/04

(52) U.S. Cl. CPC C07D 471/14; A61K 31/4375; A61K 31/4439; A61K 31/444; A61K 31/501; A61K 31/519; C07D 401/04; C07D 401/14; C07D 403/04; C07D 409/14; C07D 487/04

(57) ABSTRACT

The present invention relates to novel substituted 1H-pyrazole-4-carboxamide compounds as SARM1 inhibitors, to pharmaceutical compositions comprising the compounds and to methods of using the compounds and compositions to treat and prevent pathological conditions involving axonal degeneration.

The present invention relates to novel SARM1 inhibitors, to pharmaceutical compositions comprising the compounds and to methods of using the compounds and compositions to treat and prevent pathological conditions involving axonal degeneration.

Axonal degeneration is a major feature of pathological conditions such as Alzheimer's disease, Parkinson's disease, amyotrophic lateral sclerosis (ALS), multiple sclerosis (MS), diabetic peripheral neuropathy, chemotherapy-induced peripheral neuropathy, inherited neuropathy, traumatic brain injury, and glaucoma. These conditions affect millions of patients and present a significant financial burden worldwide.

Sterile Alpha and Toll/Interleukin receptor-1 (TIR) motif-containing 1 (SARM1) has been identified as the central executioner in the injury-induced axon death pathway known as Wallerian degeneration (O'Neill, L. A. & Bowie, A. G., Nat. Rev. Immunol., 2007, 7, 353-364; Osterloh, J. M., et al., Science, 2012, 337, 481-484; Gerdts, J., et al., J. Neurosci. 33, 2013, 13569-13580). Mechanistic studies have revealed that activation of SARM1 via axonal injury or forced dimerization of SARM1-TIR domains promotes rapid depletion of nicotinamide adenine dinucleotide (NAD+), resulting in axonal degradation (Gerdts, J., et al., Science, 2015, 348, 453-457). Genetic knockout of SARM1 allows for preservation of axons for fourteen or more days after nerve transection (Osterloh, J. M., et al., Science, 2012, 337, 481-484; Gerdts, J., et al. J. Neurosci., 2013, 33, 13569-13580) and improves functional outcomes in mice after traumatic brain injury (Henninger, N. et al., Brain 139, 2016, 1094-1105). In addition to the role of SARM1 in direct axonal injury, SARM1 is also required for axonal degeneration observed in conditions such as chemotherapy-induced peripheral neuropathy. Loss of SARM1 blocks chemotherapy-induced peripheral neuropathy, inhibiting both axonal degeneration and heightened pain sensitivity that develops after chemotherapeutic vincristine treatment (Geisler et al, Brain, 2016, 139, 3092-3108).

WO 2021/142006 A1 discloses certain compounds and methods useful for inhibiting SARM1 and/or treating and/or preventing axonal degeneration.

Currently there are no approved medications for the treatment and/or prevention of axonal degeneration. There is an unmet need for potent SARM1 inhibitors possessing improved metabolic profiles for the treatment and prevention of pathological conditions involving axonal degeneration.

The present invention provides novel SARM1 inhibitors for use in the treatment and prevention of pathological conditions involving axonal degeneration. In addition, the present invention provides novel SARM1 inhibitors that possess increased potency and that are subject to reduced metabolic clearance.

Accordingly, the present invention provides a compound of Formula I:

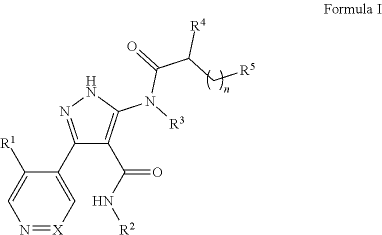

wherein
X is selected from CH and N;
R¹, R², R³ and R⁴ are each independently selected from H and C₁₋₄ alkyl, wherein R¹ and R² may optionally form a 6-membered heterocyclic ring together with the atoms they are attached to, wherein R² and R³ may optionally form a 6- to 7-membered heterocyclic ring together with the atoms they are attached to, and wherein R³ and R⁴ may optionally form a 5- to 6-membered heterocyclic ring together with the atoms they are attached to;
n is 0, 1 or 2;
R⁵ is selected from phenyl optionally substituted with 1 to 3 R^{m} and 5- to 6-membered heteroaryl containing 1 to 3 heteroatoms selected from oxygen, nitrogen and sulfur and optionally substituted with 1 to 3 R^{m}; and
R^{m} is selected from halogen, cyano, C₁₋₄ alkyl, trihalomethyl and —OC₁₋₄ alkyl; or a pharmaceutically acceptable salt thereof.

In an embodiment R¹, R², R³ and R⁴ are each independently selected from H and C₁₋₄ alkyl.

In an embodiment the compound is of Formula II:

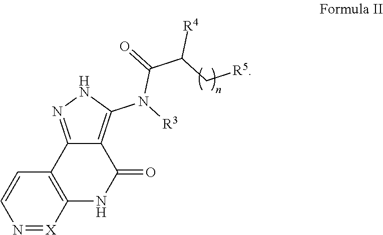

In another embodiment the compound is of Formula III:

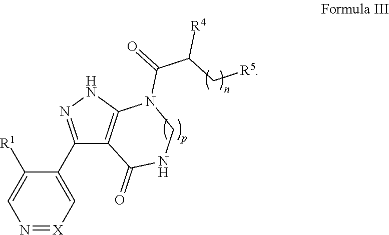

In a further embodiment the compound is of Formula IV:

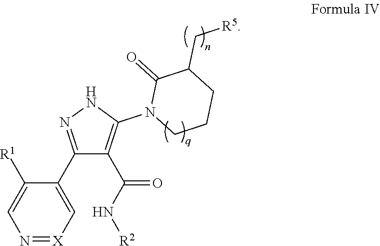

In an embodiment of the invention, X is CH. In another embodiment, X is N.

In an embodiment of invention R¹, R², R³ and R⁴ are each independently selected from H, methyl, ethyl, n-propyl and iso-propyl.

In an embodiment of the invention n is 0 or 1. In another embodiment n is 1.

In an embodiment of the invention p is 1.

In an embodiment of the invention q is 0. In another embodiment q is 1.

In an embodiment of the invention R⁵ is phenyl. In another embodiment R⁵ is phenyl substituted with 1 to 3 R^{m}. In a further embodiment R⁵ is 5- to 6-membered heteroaryl containing 1 to 3 heteroatoms selected from oxygen, nitrogen, and sulfur. In yet a further embodiment R⁵ is 5- to 6-membered heteroaryl containing 1 to 3 heteroatoms selected from oxygen, nitrogen and sulfur and substituted with 1 to 3 R^{m}. In another embodiment R⁵ is 5- to 6-membered heteroaryl containing 1 to 3 nitrogen atoms. In a further embodiment R⁵ is 5- to 6-membered heteroaryl containing 1 to 3 nitrogen atoms and substituted with 1 to 3 R^{m}. In yet a further embodiment R⁵ is pyridine. In another embodiment R⁵ is pyridine substituted with 1 to 3 R^{m}.

In an embodiment of the invention each R™ is independently selected from F, Cl, Br, I, cyano, and trifluoromethyl.

In an embodiment of the invention R⁵ is selected from the group consisting of:

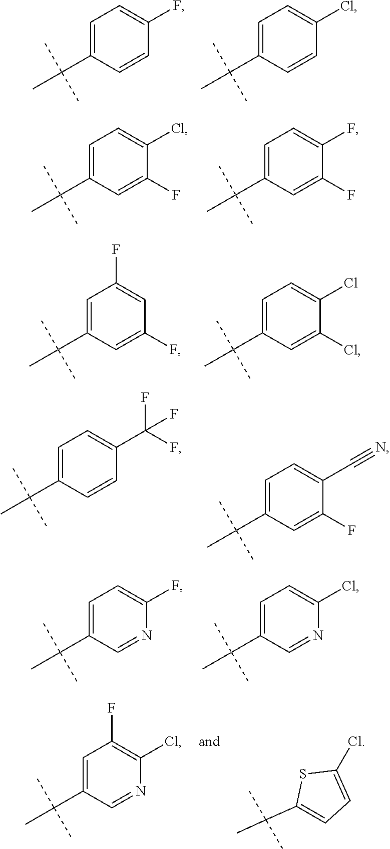

In an embodiment, the invention provides a compound selected from:

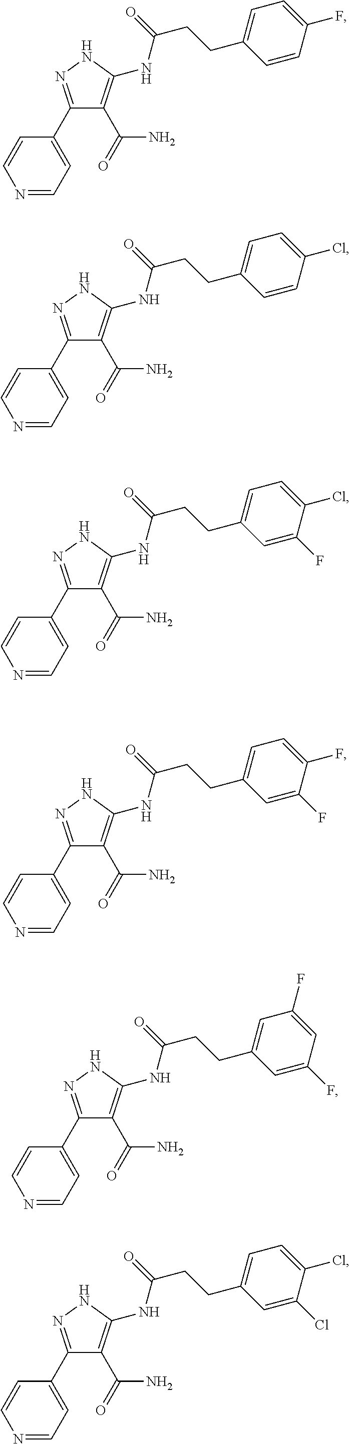 [SMILES: NC(=O)c1c(-c2ccncc2)n[nH]c1NC(=O)CCc1ccc(F)cc1 ; NC(=O)c1c(-c2ccncc2)n[nH]c1NC(=O)CCc1ccc(Cl)cc1 ; NC(=O)c1c(-c2ccncc2)n[nH]c1NC(=O)CCc1ccc(Cl)c(F)c1 ; NC(=O)c1c(-c2ccncc2)n[nH]c1NC(=O)CCc1ccc(F)c(F)c1 ; NC(=O)c1c(-c2ccncc2)n[nH]c1NC(=O)CCc1cc(F)cc(F)c1 ; NC(=O)c1c(-c2ccncc2)n[nH]c1NC(=O)CCc1ccc(Cl)c(Cl)c1]

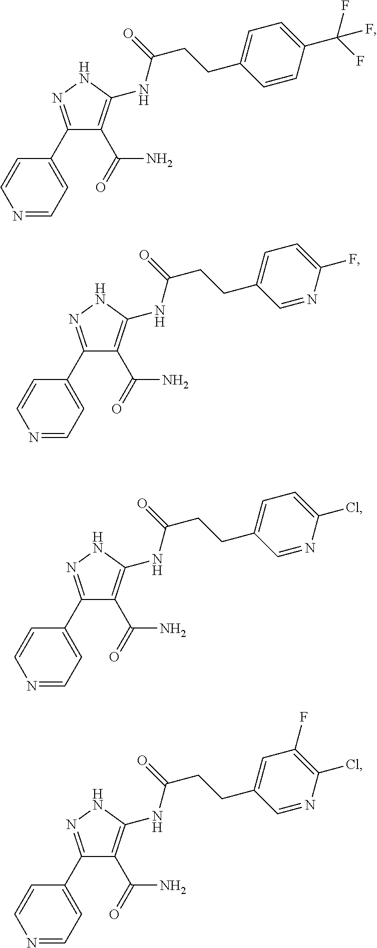 [SMILES: NC(=O)c1c(-c2ccncc2)n[nH]c1NC(=O)CCc1ccc(C(F)(F)F)cc1 ; NC(=O)c1c(-c2ccncc2)n[nH]c1NC(=O)CCc1ccc(F)nc1 ; NC(=O)c1c(-c2ccncc2)n[nH]c1NC(=O)CCc1ccc(Cl)nc1 ; NC(=O)c1c(-c2ccncc2)n[nH]c1NC(=O)CCc1cnc(Cl)c(F)c1]

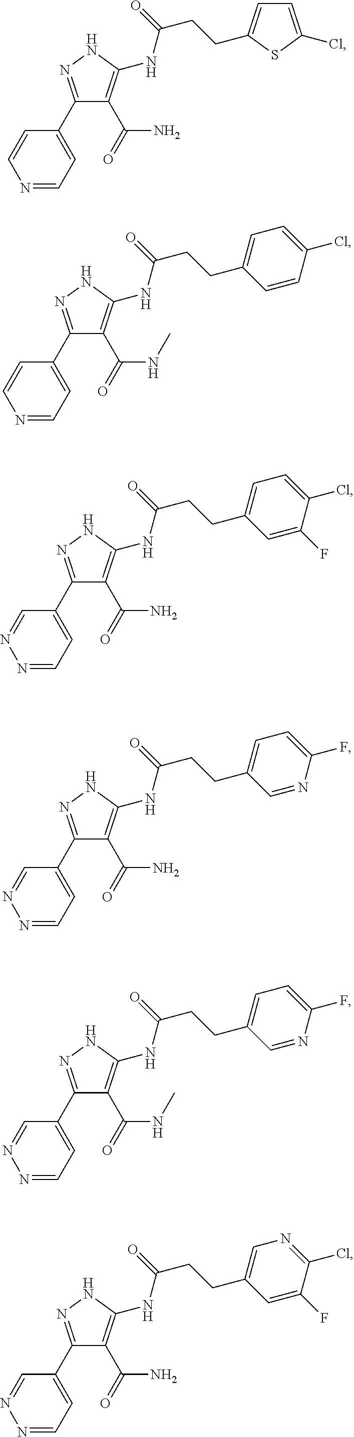 [SMILES: NC(=O)c1c(-c2ccncc2)n[nH]c1NC(=O)CCc1ccc(Cl)s1 ; CNC(=O)c1c(-c2ccncc2)n[nH]c1NC(=O)CCc1ccc(Cl)cc1 ; NC(=O)c1c(-c2ccnnc2)n[nH]c1NC(=O)CCc1ccc(Cl)c(F)c1 ; NC(=O)c1c(-c2ccnnc2)n[nH]c1NC(=O)CCc1ccc(F)nc1 ; CNC(=O)c1c(-c2ccnnc2)n[nH]c1NC(=O)CCc1ccc(F)nc1 ; NC(=O)c1c(-c2ccnnc2)n[nH]c1NC(=O)CCc1cnc(Cl)c(F)c1]

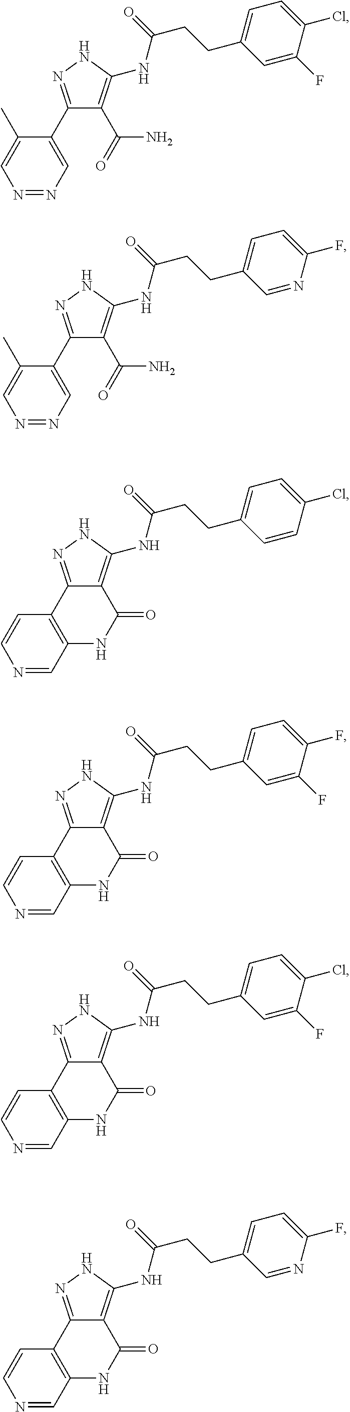 [SMILES: Cc1cnncc1-c1n[nH]c(NC(=O)CCc2ccc(Cl)c(F)c2)c1C(N)=O ; Cc1cnncc1-c1n[nH]c(NC(=O)CCc2ccc(F)nc2)c1C(N)=O ; O=C(CCc1ccc(Cl)cc1)Nc1[nH]nc2c1c(=O)[nH]c1cnccc12 ; O=C(CCc1ccc(F)c(F)c1)Nc1[nH]nc2c1c(=O)[nH]c1cnccc12 ; O=C(CCc1ccc(Cl)c(F)c1)Nc1[nH]nc2c1c(=O)[nH]c1cnccc12 ; O=C(CCc1ccc(F)nc1)Nc1[nH]nc2c1c(=O)[nH]c1cnccc12]

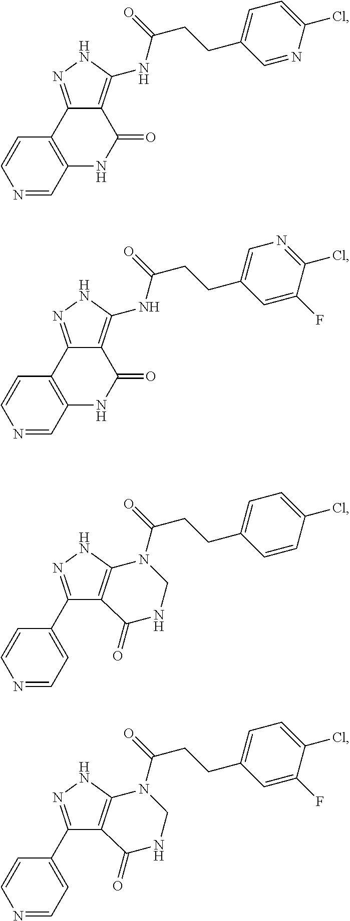 [SMILES: O=C(CCc1ccc(Cl)nc1)Nc1[nH]nc2c1c(=O)[nH]c1cnccc12 ; O=C(CCc1cnc(Cl)c(F)c1)Nc1[nH]nc2c1c(=O)[nH]c1cnccc12 ; O=C1NCN(C(=O)CCc2ccc(Cl)cc2)c2[nH]nc(-c3ccncc3)c21 ; O=C1NCN(C(=O)CCc2ccc(Cl)c(F)c2)c2[nH]nc(-c3ccncc3)c21]

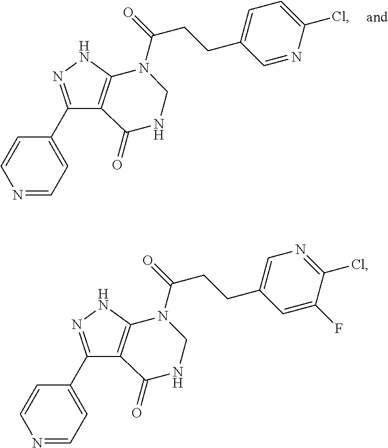 [SMILES: O=C1NCN(C(=O)CCc2ccc(Cl)nc2)c2[nH]nc(-c3ccncc3)c21 ; O=C1NCN(C(=O)CCc2cnc(Cl)c(F)c2)c2[nH]nc(-c3ccncc3)c21]

or a pharmaceutically acceptable salt thereof.

The present invention provides a pharmaceutical composition comprising a compound or a pharmaceutically acceptable salt thereof according to any of the above embodiments, with one or more pharmaceutically acceptable carriers, diluents, or excipients.

The present invention provides a method of treating or preventing a disease associated with axonal degeneration in a patient, comprising administering to a patient in need of such treatment an effective amount of a compound or pharmaceutically acceptable salt thereof, or pharmaceutical composition thereof, according to any of the above embodiments.

The present invention also provides a method of treating or preventing a disease associated with SARM1 activation in a patient, comprising administering to a patient in need of such treatment an effective amount of a compound or pharmaceutically acceptable salt thereof, or pharmaceutical composition thereof, according to any of the above embodiments.

The present invention also provides a method of treating or preventing a disease selected from amyotrophic lateral sclerosis (ALS), multiple sclerosis (MS), diabetic neuropathy and chemotherapy-induced peripheral neuropathy in a patient, comprising administering to a patient in need of such treatment an effective amount of a compound or pharmaceutically acceptable salt thereof, or pharmaceutical composition according to any of the above embodiments.

The present invention provides a compound or a pharmaceutically acceptable salt thereof according to any one of the above embodiments for use in therapy.

Furthermore, the present invention provides a compound or a pharmaceutically acceptable salt thereof according to any one of the above embodiments for use in the treatment or prevention of a disease associated with axonal degeneration.

In addition, the present invention provides a compound or a pharmaceutically acceptable salt thereof according to any one of the above embodiments for use in the treatment or prevention of a disease selected from amyotrophic lateral sclerosis (ALS), multiple sclerosis (MS), diabetic neuropathy and chemotherapy-induced peripheral neuropathy.

The present invention provides the use of a compound or pharmaceutically acceptable salt thereof according to any one of the above embodiments for the manufacture of a medicament for the treatment or prevention of a disease associated with axonal degeneration.

In addition, the present invention provides the use of a compound or pharmaceutically acceptable salt thereof, according to any one of the above embodiments for the manufacture of a medicament for the treatment or prevention of a disease selected from amyotrophic lateral sclerosis (ALS), multiple sclerosis (MS), diabetic neuropathy and chemotherapy-induced peripheral neuropathy.

As used herein, the term “alkyl”, used alone or as part of a larger moiety, refers to a saturated, straight, or branched chain hydrocarbon group containing one or more carbon atoms.

As used herein, the term “heteroaryl”, refers to a cyclic aromatic group containing one or more heteroatoms.

As used herein, the term “heteroatom” refers to nitrogen, oxygen, or sulfur, and includes any oxidized form of nitrogen or sulfur, and any quaternized form of a basic nitrogen.

As used herein, the term “heterocyclic” refers to a cyclic saturated group containing carbon atoms and one or more heteroatoms.

As used herein, the term “patient” refers to a human.

As used herein, the term “preventing” refers to preventing the occurrence of a disease or averting resulting complications after its onset.

As used herein, the term “treating” includes slowing, stopping, or reversing the progression or severity of an existing symptom or disorder.

As used herein, the term “effective amount” refers to the amount or dose of compound of the invention, or a pharmaceutically acceptable salt thereof which, upon single or multiple dose administration to the patient, provides the desired effect in the patient under diagnosis or treatment.

The compounds of the present invention are preferably formulated as pharmaceutical compositions administered by any route which makes the compound bioavailable, including oral and transdermal routes. Most preferably, such compositions are for oral administration. Such pharmaceutical compositions and processes for preparing same are well known in the art (See, e.g., Remington: The Science and Practice of Pharmacy, A. Adejare, Editor, 23rd Edition, Elsevier Academic Press, 2020).

The compounds of the present invention, or pharmaceutically acceptable salts thereof, may be prepared according to the following Preparations and Examples by methods well known and appreciated in the art. Suitable reaction conditions for the steps of these Preparations and Examples are well known in the art and appropriate substitutions of solvents and co-reagents are within the skill of the art. Likewise, it will be appreciated by those skilled in the art that synthetic intermediates may be isolated and/or purified by various well-known techniques as needed or desired, and that frequently, it will be possible to use various intermediates directly in subsequent synthetic steps with little or no purification. As an illustration, compounds of the preparations and examples can be isolated, for example, by silica gel purification, isolated directly by filtration, or crystallization. Furthermore, the skilled artisan will appreciate that in some circumstances, the order in which moieties are introduced is not critical. The particular order of steps required to produce the compounds of the present invention is dependent upon the particular compound being synthesized, the starting compound, and the relative liability of the substituted moieties, and is well appreciated by the skilled chemist. All substituents, unless otherwise indicated, are as previously defined, and all reagents are well known and appreciated in the art.

Certain abbreviations are defined as follows: “AcONa” refers to sodium acetate; “Ar” refers to heteroaryl or aryl; “DCM” refers to dichloromethane; “DIPEA” refers to N, N-diisopropylethylamine; “DMA” refers to dimethylacetamide; “DMF” refers to dimethylformamide; “DIPEA” refers to N, N-diisopropylethylamine; “Et3N” refers to triethylamine; “EtOAc” refers to ethyl acetate; “EtOH” stands for ethyl alcohol; “EtONa” refers to sodium ethoxide; HATU refers to (1-[bis(dimethylamino)methylene]-1H-1,2,3-triazolo[4,5-b]pyridinium 3-oxide hexafluorophosphate; “HPLC” refers to high-performance liquid chromatography; “hr.” or “hrs.” refers to hour or hours; “LDA” refers to lithium diisopropylamide; “LiHMDS” refers to lithium bis(trimethylsilyl)amide; “MeCN” refers to acetonitrile; “MeOH” refers to methanol; “min” refers to minute or minutes; “NBS” refers to N-bromosuccinimide; “PdC12.dppf” refers to 1,1′-bis(diphenylphosphino) ferrocene dichloropalladium (II); “Prep-HPLC” refers to preparative HPLC; “SEM-CI” refers to 2-(trimethylsilyl) ethoxymethyl chloride; “TFA” refers to trifluoroacetic acid; “TfOH” refers to trifluoromethanesulfonic acid; “THF” refers to tetrahydrofuran; and “T3P” refers to propanephosphonic acid anhydride.

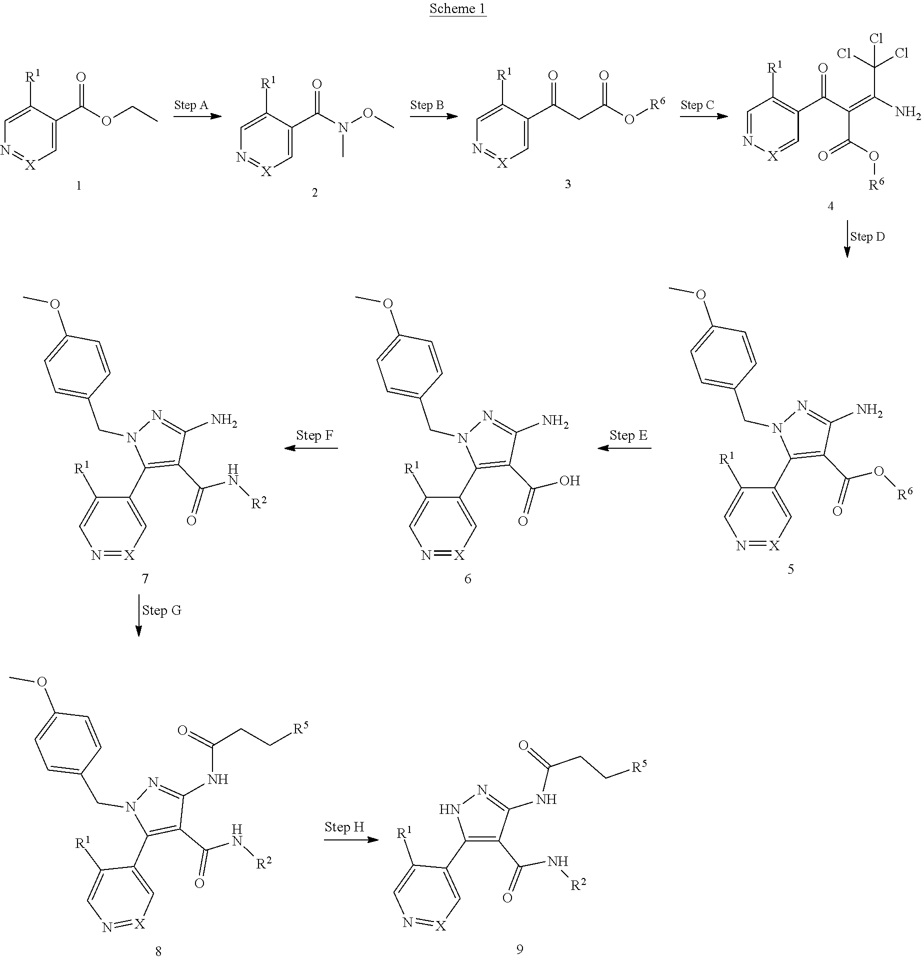

Scheme 1, step A depicts the formation of a Weinreb amide from compound (1) using N,O-dimethylhydroxylamine hydrochloride in a solvent such as THF with a base such as LiHMDS to give compound (2). Step B shows the addition of EtOAc to compound (2) using a base such as LDA in a solvent such as THF to give compound (3). Step C shows the addition of trichloroacetonitrile to compound (3) using a base such as sodium acetate trihydrate in a solvent such as EtOH to give compound (4). Step D depicts the cyclization of compound (4) with (4-methoxybenzyl) hydrazine hydrochloride using a base such as TEA in a solvent such as EtOH to give compound (5). In cases where R⁶ is ethyl, the basic hydrolysis of compound (5) using NaOH in a solvent system such as THE, EtOH, and water to give compound (6) is shown in step E. Alternatively, when R⁶ is t-butyl, step E shows an acidic deprotection of compound (5) with an acid such as TFA in a solvent such as DCM to give compound (6). Step F shows the amide coupling of compound (6) with either ammonium chloride or methylamine hydrochloride using a coupling reagent such as HATU and a base such as DIPEA in a solvent such as DMF to give compound (7). One skilled in the art will recognize that many other amide coupling reagents, bases, and solvents could be used to perform this coupling. Step G shows the amide coupling of compound (7) with an aryl propanoic acid using a coupling reagent such as HATU and a base such as DIPEA in a solvent such as DMA to give compound (8). One skilled in the art will recognize that many other amide coupling reagents, bases, and solvents could be used to perform this coupling. Step H depicts the acidic deprotection of compound (8) with an acid such as TFA in a solvent such as DCM to give compound (9).

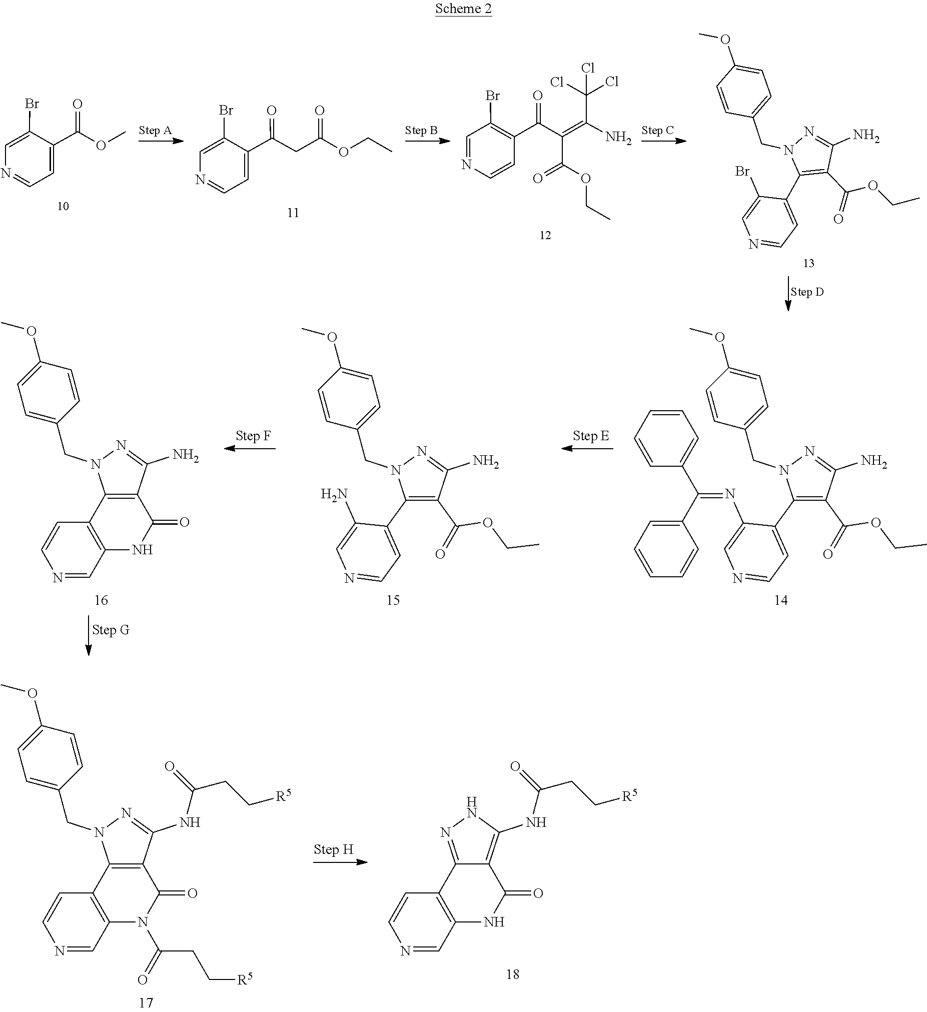

Scheme 2, step A depicts the addition of EtOAc to compound (10) using a base such as LDA in a solvent such as THF to give compound (11). Steps B and C are essentially analogous to those found in Scheme 1, steps C and D and show the conversion of compound (11) to compound (12) and then compound (13). Step D shows the coupling of compound (13) and diphenylmethanimine using a catalyst-ligand system such as Pd₂(dba)₃ and (9,9-dimethyl-9H-xanthene-4,5-diyl)bis(diphenylphosphane) with a base such a cesium carbonate in a solvent such as DMF to give compound (14). The acidic deprotection of compound (14) with an acid such as TFA in a solvent such as DCM to give compound (15) is shown in step E. Step F shows the intramolecular cyclization of compound (15) to compound (16) through use of a base such as sodium ethoxide in a solvent such as EtOH. Step G shows the amide coupling of compound (16) with an aryl propanoic acid using a coupling reagent such as T3P and a base such as DIPEA in a solvent such as DMA to give compound (17). One skilled in the art will recognize that many other amide coupling reagents, bases, and solvents could be used to perform this coupling. Step H shows a two step deprotection of compound (17). The first step uses a base such as potassium carbonate in a solvent such as MeOH with heating to remove the amide from the inner core nitrogen. This is followed by treatment with an acid such as TFA in a solvent such as DCM to give compound (18).

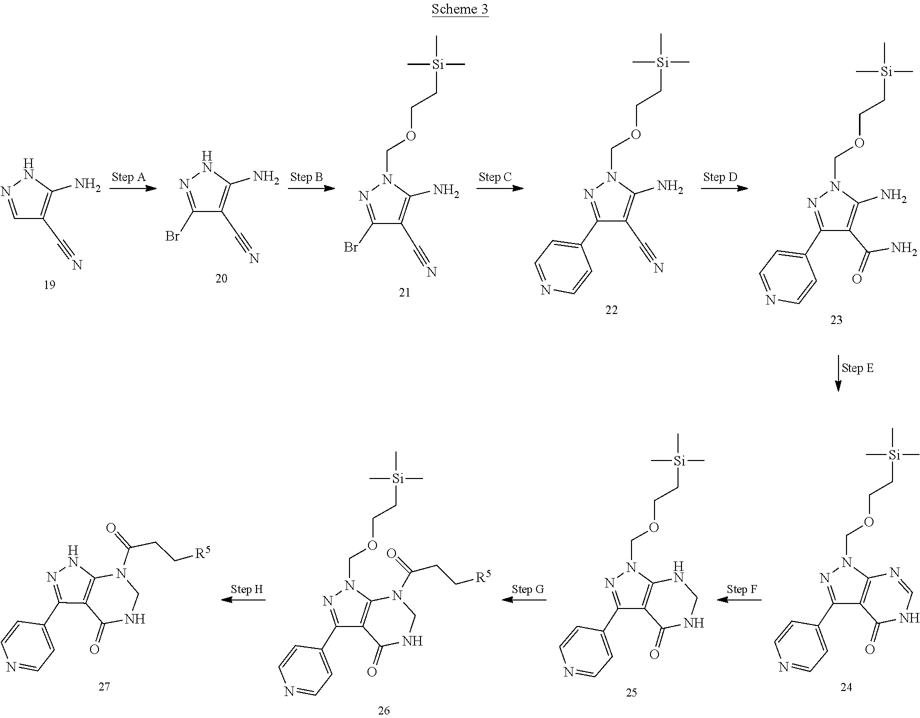

Scheme 3, step A depicts the bromination of compound (19) with NBS in a solvent such as DMF to give compound (20). Step B shows the reaction of compound (20) with SEM-Cl using a base such as NaH in a solvent such as DMF to give compound (21). Step C shows a Suzuki coupling between compound (21) and 4-pyridylboronic acid using a catalyst such as PdCl₂.dppf and a base such as potassium carbonate in a solvent system such as dioxane and water to give compound (22). One skilled in the art will recognize that there are many catalyst, ligand, base, and solvent combinations which may be utilized to perform this type of coupling. Step D shows the conversion of the nitrile of compound (22) to a primary amide through treatment with a base such as KOH in a solvent system such as EtOH and water to give compound (23). Step E shows the cyclization of compound (23) with formaldehyde in a solvent such as toluene to give compound (24). Step F shows the reduction of compound (24) with a suitable reducing agent such as NaBH₄ in a solvent such as EtOH to give compound (25). The conversion of compound (25) to compound (26) in step G is essentially analogous to Scheme 2, step G. The conversion of compound (26) to compound (27) in step H is essentially analogous to Scheme 1, step H.

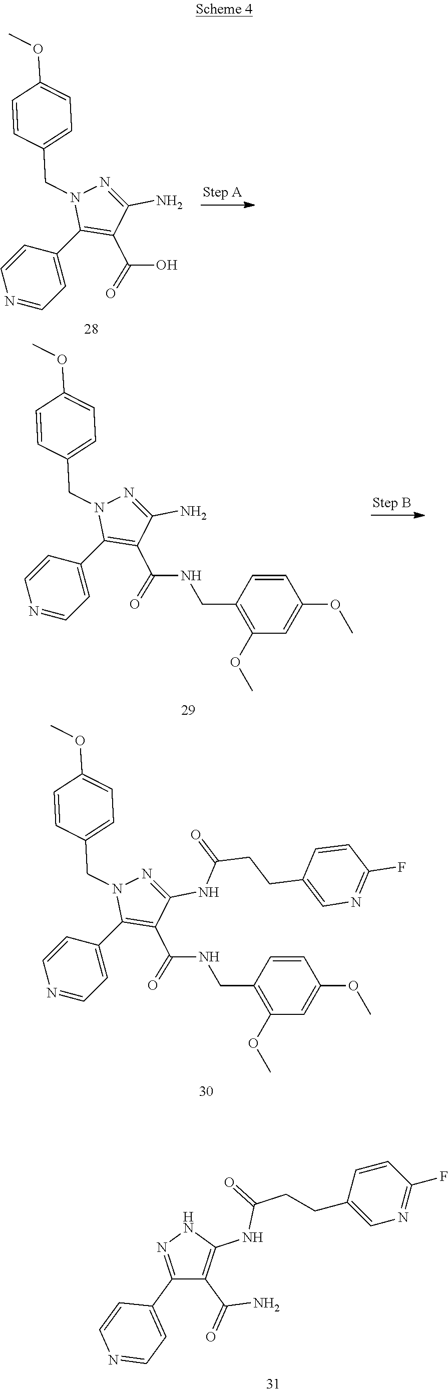

Scheme 4 step A shows the amide coupling between compound (28) and (2,4-dimethoxyphenyl) methanamine using a coupling reagent such as HATU and a base such as DIPEA in a solvent such as DMF to give compound (29). One skilled in the art will recognize that many other amide coupling reagents, bases, and solvents could be used to perform this coupling. The conversion of compound (29) to compound (30) in step G is essentially analogous to Scheme 2, step G. Step C shows the global deprotection of compound (30) using an appropriate acid system such as TFA and TfOH in a solvent such as DCM to give compound (31).

### Preparation 1

### N-Methoxy-N,5-dimethylpyridazine-4-carboxamide

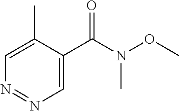

A mixture of ethyl 5-methylpyridazine-4-carboxylate (1.2 g, 7.23 mmol) and N,O-dimethylhydroxylamine hydrochloride (1.42 g, 14.46 mmol) in THF (20 mL) was stirred at −70° C. for 5 min. LiHMDS (1.0M in THF, 50.6 mL, 50.6 mmol) was added slowly, and stirred at −70° C. for 2.0 hrs. The reaction mixture was poured into saturated aqueous NH₄Cl solution (200 mL) and extracted with DCM (50 mL×3). The combined organic layers were dried over Na₂SO₄ and concentrated in vacuo. The crude material was purified via silica gel flash chromatography eluting with 3% petroleum ether in EtOAc to give the title compound (0.94 g, 71.2%) as a yellow oil. ES/MS m/z=182 (M+H).

### Preparation 2

### Ethyl 3-(5-methylpyridazin-4-yl)-3-oxo-propanoate

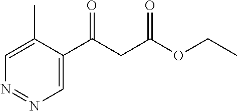

To a mixture of EtOAc (1.37 g, 15.6 mmol) in THF (20 mL) was added LDA (1M in THF, 15.6 mL) slowly at −70° C. then stirred for 1.5 hrs. N-Methoxy-N,5-dimethylpyridazine-4-carboxamide (0.94 g, 5.2 mmol) in THF (8 mL) was added slowly and stirred at −70° C. for 2.0 hrs. The reaction mixture was poured into saturated aqueous NH₄Cl solution (200 mL) and extracted with DCM (50 mL×3). The combined organic layers were dried over Na₂SO₄ and concentrated in vacuo. The crude material was purified via silica gel flash chromatography eluting with 50% petroleum ether in EtOAc to give the title compound (0.53 g, 49.1%) as a yellow oil. ES/MS m/z=209 (M+H).

The compound in Table 1 were prepared in a manner essentially analogous to that found in Preparation 2.

TABLE 1

| Prep | | | |
|---|---|---|---|
| No | Chemical Name | Structure | ES/MS m/z |
| 3 | Ethyl 3-oxo-3- (pyridazin-4- y1)propanoate | 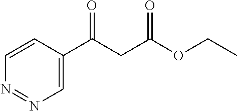 | 195.0 (M + H) |

### Preparation 4

### Ethyl 3-(3-bromopyridin-4-yl)-3-oxopropanoate

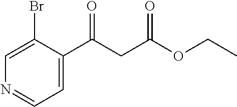

LDA solution (1M in THF, 32 mL) was slowly added at −60° C., over 1 hr. to a mixture of EtOAc (4.2 g, 48 mmol) in THF (80 mL). Methyl 3-bromoisonicotinate (7.0 g, 32 mmol) in THF (20 mL) was added slowly, and the mixture stirred at −60° C. for 2.0 hrs. Saturated aqueous NH₄Cl solution (30 mL) was added to the reaction mixture and then extracted with EtOAc (150 mL×2). The combined organic layers were dried over Na₂SO₄ and concentrated in vacuo to give crude product. The crude product was purified via silica gel flash chromatography eluting with 20:1 petroleum ether:EtOAc to give the title compound (10 g, 63%) as a yellow oil. ES/MS m/z=274 (M+H).

### Preparation 5

### Ethyl (Z)-3-amino-4,4,4-trichloro-2-(5-methylpyridazine-4-carbonyl) but-2-enoate

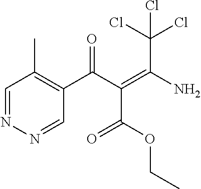 [SMILES: CCOC(=O)/C(C(=O)c1cnncc1C)=C(\N)C(Cl)(Cl)Cl]

To a mixture of ethyl 3-(5-methylpyridazin-4-yl)-3-oxo-propanoate (530 mg, 2.55 mmol) in EtOH (10 mL) was added trichloroacetonitrile (404 mg, 2.81 mmol) and sodium acetate trihydrate (251 mg, 3.06 mmol). The mixture was stirred at ambient temperature for 2.0 hrs. The reaction mixture was concentrated in vacuo to give the title compound (1.08 g, crude) as a yellow oil. ES/MS m/z=352 (M+H).

The compounds in Table 2 were prepared in a manner essentially analogous to that found in Preparation 5.

TABLE 2

| Prep | | | |
|---|---|---|---|
| No | Chemical Name | Structure | ES/MS m/z |
| 6 | Ethyl (Z)-3-amino- 4,4,4-trichloro-2- (pyridazine-4- carbonyl)but-2- enoate | 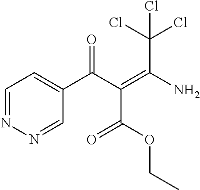 [SMILES: CCOC(=O)/C(C(=O)c1ccnnc1)=C(\N)C(Cl)(Cl)Cl] | 337 (M + H) |
| 7 | Ethyl (Z)-3-amino-2- (3- bromoisonicotinoyl)- 4,4,4-trichlorobut-2- enoate | 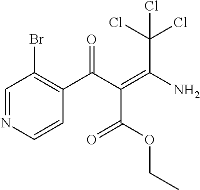 | 419 (M + H) |

### Preparation 8

### tert-Butyl (Z)-3-amino-4,4,4-trichloro-2-isonicotinoylbut-2-enoate

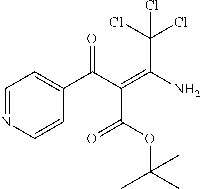 [SMILES: CC(C)(C)OC(=O)/C(C(=O)c1ccncc1)=C(\N)C(Cl)(Cl)Cl]

To a mixture of tert-butyl 3-oxo-3-(pyridin-4-yl)propanoate (prepared as described in Bioorg. Med. Chem. Lett., 2015, 25, 3810) (29.0 g, 0.13 mol) in EtOH (150 mL) was added trichloroacetonitrile (20.0 g, 0.14 mmol) and sodium acetate trihydrate (11.5 g, 0.14 mmol). The reaction was stirred at ambient temperature for 2 hrs. The reaction mixture was used directly without further workup. ES/MS m/z=367 (M+H).

### Preparation 9

### Ethyl 3-amino-1-(4-methoxybenzyl)-5-(5-methylpyridazin-4-yl)-1H-pyrazole-4-carboxylate

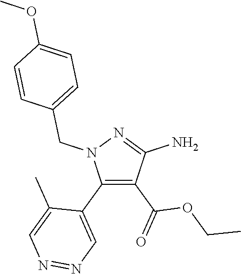 [SMILES: CCOC(=O)c1c(N)nn(Cc2ccc(OC)cc2)c1-c1cnncc1C]

To a mixture of ethyl (Z)-3-amino-4,4,4-trichloro-2-(5-methylpyridazine-4-carbonyl) but-2-enoate (1.08 g, crude) in EtOH (10 mL) was added TEA (0.77 g, 7.6 mmol) and (4-methoxybenzyl) hydrazine hydrochloride (0.58 g, 3.05 mmol). The mixture was stirred at 65° C. for 2 hrs. then concentrated in vacuo. The crude was purified via silica gel flash chromatography eluting with 3% petroleum ether in EtOAc to give the title compound (0.54 g, 58.1%) as a yellow brown oil. ES/MS m/z=368 (M+H).

The compounds in Table 3 were prepared in a manner essentially analogous to that found in Preparation 9.

TABLE 3

| Prep | | | |
|---|---|---|---|
| No | Chemical Name | Structure | ES/MS m/z |
| 10 | Ethyl 3-amino-1-(4- methoxybenzyl)-5-(pyridazin-4- y1)-1H-pyrazole-4-carboxylate | 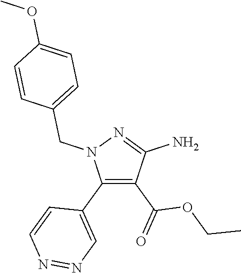 [SMILES: CCOC(=O)c1c(N)nn(Cc2ccc(OC)cc2)c1-c1ccnnc1] | 355 (M + H) |
| 11 | Ethyl 3-amino-5-(3- bromopyridin-4-y1)-1-(4- methoxybenzyl)-1H-pyrazole-4- carboxylate | 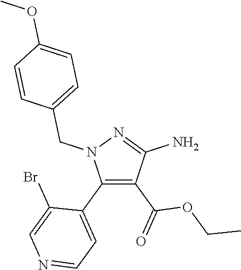 [SMILES: CCOC(=O)c1c(N)nn(Cc2ccc(OC)cc2)c1-c1ccncc1Br] | 431 (M + H) |

### Preparation 12

### tert-Butyl 3-amino-1-(4-methoxybenzyl)-5-(pyridin-4-yl)-1H-pyrazole-4-carboxylate

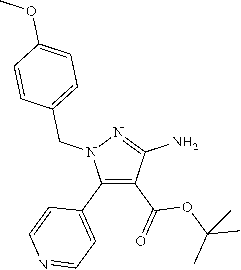 [SMILES: COc1ccc(Cn2nc(N)c(C(=O)OC(C)(C)C)c2-c2ccncc2)cc1]

To the mixture of tert-butyl (Z)-3-amino-4,4,4-trichloro-2-isonicotinoylbut-2-enoate was added (4-methoxybenzyl) hydrazine hydrochloride (30.0 g, 0.16 mol) and TEA (23.4 g, 0.39 mmol). The mixture was stirred at ambient temperature for 2 hrs. The mixture was then filtered, and the resulting solid was dried in vacuo to give the title compound (30.0 g, 60%) as a white solid. ES/MS m/z=381 (M+H)

### Preparation 13

### 3-Amino-1-(4-methoxybenzyl)-5-(5-methylpyridazin-4-yl)-1H-pyrazole-4-carboxylic acid

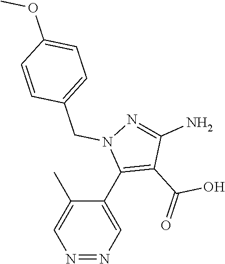 [SMILES: COc1ccc(Cn2nc(N)c(C(=O)O)c2-c2cnncc2C)cc1]

To a mixture of ethyl 3-amino-1-(4-methoxybenzyl)-5-(5-methylpyridazin-4-yl)-1H-pyrazole-4-carboxylate (540 mg, 1.47 mmol) in THF (4 mL), EtOH (4 mL), and water (2 mL) was added NaOH (118 mg, 2.94 mmol). The mixture was stirred at 75° C. for 4 hrs. The mixture was then concentrated in vacuo. The mixture was adjusted to pH=3~4 with 1M HCl. The resulting slurry was filtered, and the filter cake washed with 20 mL of ice water. The title compound (350 mg, 70.1%) was obtained as a yellow solid. ES/MS m/z=340 (M+H).

The compound in Table 4 was prepared in a manner essentially analogous to that found in Preparation 13.

TABLE 4

| Prep | | | |
|---|---|---|---|
| No | Chemical Name | Structure | ES/MS m/z |
| 14 | 3-Amino-1-(4-methoxybenzyl)- 5-(pyridazin-4-y1)-1H-pyrazole- 4-carboxylic acid | 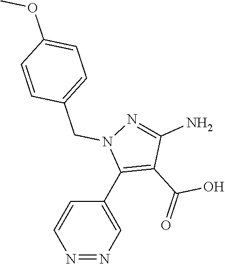 [SMILES: COc1ccc(Cn2nc(N)c(C(=O)O)c2-c2ccnnc2)cc1] | 326.7 (M + H) |

### Preparation 15

### 3-Amino-1-(4-methoxybenzyl)-5-(pyridin-4-yl)-1H-pyrazole-4-carboxylic acid

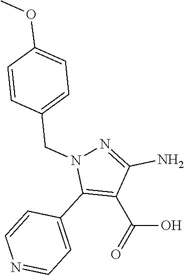 [SMILES: COc1ccc(Cn2nc(N)c(C(=O)O)c2-c2ccncc2)cc1]

To a mixture of tert-butyl 3-amino-1-(4-methoxybenzyl)-5-(pyridin-4-yl)-1H-pyrazole-4-carboxylate (27.0 g, 0.08 mmol) in DCM (100 mL) was added TFA (100 mL).

The mixture was stirred at ambient temperature for 3 hrs. The reaction mixture was concentrated in vacuo followed by the addition of water (50 mL) and saturated NaHCO₃ (150 mL). The resulting precipitate was filtered and dried in vacuo to give the title compound (12 g, 52%) as a white solid. ES/MS m/z=325 (M+H).

### Preparation 16

### 3-Amino-1-(4-methoxybenzyl)-5-(5-methylpyridazin-4-yl)-1H-pyrazole-4-carboxamide

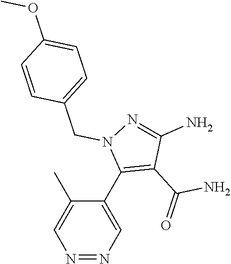 [SMILES: COc1ccc(Cn2nc(N)c(C(N)=O)c2-c2cnncc2C)cc1]

A mixture of 3-amino-1-(4-methoxybenzyl)-5-(5-methylpyridazin-4-yl)-1H-pyrazole-4-carboxylic acid (350 mg, 1.03 mmol), ammonium chloride (1.09 g, 20.6 mmol), HATU (783 mg, 2.06 mmol), DIPEA (399 mg, 3.09 mmol) in DMF (5 mL) was stirred at ambient temperature for 4 hrs. To the reaction mixture was added water (30 mL) which was extracted with DCM (30 mL×5). The combined organic layer was dried over Na₂SO₄ and concentrated in vacuo to give the title compound (400 mg, crude) as a brown oil. ES/MS m/z=339 (M+H).

The compounds in Table 5 were prepared in a manner essentially analogous to that found in Preparation 16.

TABLE 5

| Prep | | | |
|---|---|---|---|
| No | Chemical Name | Structure | ES/MS m/z |
| 17 | 3-Amino-1-(4-methoxybenzyl)- 5-(pyridazin-4-yl)-1H-pyrazole- 4-carboxamide | 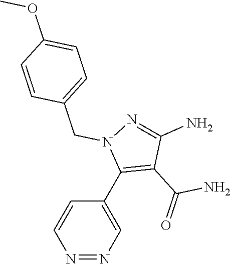 [SMILES: COc1ccc(Cn2nc(N)c(C(N)=O)c2-c2ccnnc2)cc1] | 325.7 (M + H) |
| 18 | 3-Amino-1-(4-methoxybenzyl)- 5-(pyridin-4-y1)-1H-pyrazole-4- carboxamide | 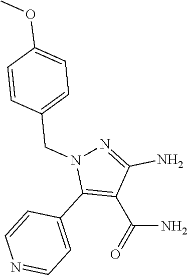 [SMILES: COc1ccc(Cn2nc(N)c(C(N)=O)c2-c2ccncc2)cc1] | 324 (M + H) |

### Preparation 19

### 3-Amino-1-(4-methoxybenzyl)-N-methyl-5-(pyridazin-4-yl)-1H-pyrazole-4-carboxamide

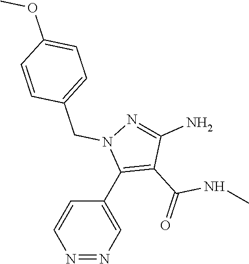

To a solution of 3-amino-1-(4-methoxybenzyl)-5-(pyridazin-4-yl)-1H-pyrazole-4-carboxylic acid (150 mg, 0.5 mmol), HATU (262 mg, 0.7 mmol) in DMF (3 mL) was added methanamine hydrochloride (46 mg, 0.7 mmol) and DIPEA (178 mg, 1.4 mmol). The reaction mixture was stirred at ambient temperature for 1.5 hrs. The mixture was diluted with water (10 mL) and extracted with EtOAc (20 mL×3). The combined organic layers were concentrated, and the crude was purified via silica gel flash chromatography eluting with 0-80% EtOAc in petroleum ether to give the title compound (130 mg, 83.3%) as a green solid. ES/MS m/z=339.7 (M+H).

### Preparation 20

### Ethyl 3-amino-5-(3-((diphenylmethylene)amino)pyridin-4-yl)-1-(4-methoxybenzyl)-1H-pyrazole-4-carboxylate

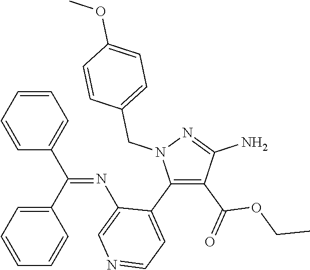 [SMILES: CCOC(=O)c1c(N)nn(Cc2ccc(OC)cc2)c1-c1ccncc1N=C(c1ccccc1)c1ccccc1]

To a mixture of ethyl 3-amino-5-(3-bromopyridin-4-yl)-1-(4-methoxybenzyl)-1H-pyrazole-4-carboxylate (1.5 g, 3.5 mmol) in DMF (10 mL) was added to Cs₂CO₃ (3.4 g, 10.5 mmol), diphenylmethanimine (1.9 g, 10.5 mmol), (9,9-dimethyl-9H-xanthene-4,5-diyl)bis(diphenylphosphane) (202 mg, 0.35 mmol), and Pd₂(dba)₃ (320 mg, 0.35 mmol). The mixture was heated by microwave at 110° C. for 8 hrs. Water (50 mL) was then added to the mixture and followed by extraction with EtOAc (100 mL×2). The combined organic layers were dried over Na₂SO₄ and concentrated in vacuo. The residue was purified via silica gel flash chromatography eluting with 20:1 petroleum ether:EtOAc to give the title compound (500 mg, 27%) as a yellow solid. ES/MS m/z=532 (M+H).

### Preparation 21

### Ethyl 3-amino-5-(3-aminopyridin-4-yl)-1-(4-methoxybenzyl)-1H-pyrazole-4-carboxylate

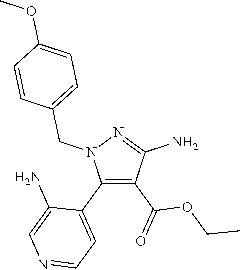 [SMILES: CCOC(=O)c1c(N)nn(Cc2ccc(OC)cc2)c1-c1ccncc1N]

TFA (1 mL) was added to a mixture of ethyl 3-amino-5-(3-((diphenylmethylene)amino)pyridin-4-yl)-1-(4-methoxybenzyl)-1H-pyrazole-4-carboxylate (500 mg, 0.94 mmol) in DCM (4 mL) and stirred at ambient temperature for 1 hr. The reaction mixture was concentrated in vacuo. Water (5 mL) and saturated NaHCO₃ (20 mL) were added to the residue and the resulting precipitate was filtered and dried in vacuo to give the title compound (200 mg, 58%) as a white solid. ES/MS m/z=368 (M+H).

### Preparation 22

### 3-Amino-1-(4-methoxybenzyl)-1,5-dihydro-4H-pyrazolo[4,3-c][1,7]naphthyridin-4-one

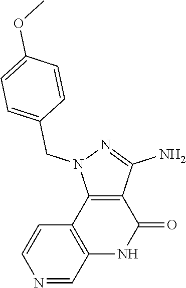 [SMILES: COc1ccc(Cn2nc(N)c3c(=O)[nH]c4cnccc4c32)cc1]

EtONa (110 mg, 1.62 mmol) was added to a mixture of ethyl 3-amino-5-(3-aminopyridin-4-yl)-1-(4-methoxybenzyl)-1H-pyrazole-4-carboxylate (200 mg, 0.54 mmol) in EtOH (10 mL) and stirred at 85° C. for 4 hrs. Water (50 mL) was added to the reaction mixture and extracted with EtOAc (100 mL×2). The combined organics were dried and concentrated in vacuo. The resulting residue was purified via silica gel flash chromatography eluting with 50% petroleum ether in EtOAc to give the title compound (140 mg, 80%) as a yellow solid. ES/MS m/z=322 (M+H).

### Preparation 23

### 5-Amino-3-bromo-1H-pyrazole-4-carbonitrile

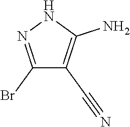

A mixture of 5-amino-1H-pyrazole-4-carbonitrile (6 g, 55.6 mmol) and NBS (11 g, 61.1 mmol) in DMF (50 mL) was stirred at ambient temperature overnight. To the reaction mixture was added water (200 mL), which was extracted with EtOAc (50 mL×3). The combined organic layers were dried over Na₂SO₄ and concentrated in vacuo. The resulting residue was purified via silica gel flash chromatography eluting with 2:3 petroleum ether:EtOAc to give the title compound (4.7 g, 45.5%) as a yellow solid. ES/MS m/z=187 (M+H).

### Preparation 24

### 5-Amino-3-bromo-1-((2-(trimethylsilyl) ethoxy)methyl)-1H-pyrazole-4-carbonitrile

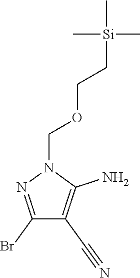 [SMILES: C[Si](C)(C)CCOCn1nc(Br)c(C#N)c1N]

To a mixture of 5-amino-3-bromo-1H-pyrazole-4-carbonitrile (4.7 g, 25.2 mmol) in DMF (40 mL) was added NaH (60% dispersion in mineral oil, 1.1 g, 27.8 mmol) at 0° C., which was then stirred at 0° C. for 30 min. SEM-Cl (5 g, 30.3 mmol) was added at 0° C., and then it was stirred while allowing to warm to ambient temperature for 2 hrs. To the reaction mixture was added water (200 mL) followed by extraction with EtOAc (50 mL×3). The combined organic layers were dried over Na₂SO₄ and concentrated in vacuo to give the title compound (6.6 g, 83.3%) as a yellow solid. ES/MS m/z=317 (M+H).

### Preparation 25

### 5-Amino-3-(pyridin-4-yl)-1-((2-(trimethylsilyl) ethoxy)methyl)-1H-pyrazole-4-carbonitrile

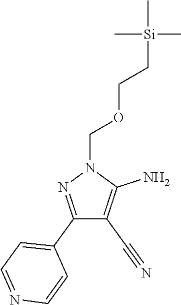 [SMILES: C[Si](C)(C)CCOCn1nc(-c2ccncc2)c(C#N)c1N]

A mixture of 5-amino-3-bromo-1-((2-(trimethylsilyl) ethoxy)methyl)-1H-pyrazole-4-carbonitrile (6.6 g, 20.9 mmol), 4-pyridylboronic acid (5.1 g, 41.8 mmol), K₂CO₃ (8.7 g, 62.7 mmol), and PdCl₂.dppf (292 mg, 0.4 mmol) in 1,4-dioxane/water (100 mL/20 mL) was stirred at 100° C. under N₂ overnight. The mixture was concentrated in vacuo, and the residue was purified via silica gel flash chromatography eluting with 2:3 petroleum ether:EtOAc to give the title compound (5 g, 76%) as a yellow solid. ES/MS m/z=316 (M+H).

### Preparation 26

### 5-Amino-3-(pyridin-4-yl)-1-((2-(trimethylsilyl) ethoxy)methyl)-1H-pyrazole-4-carboxamide

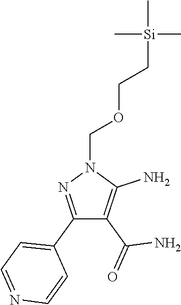 [SMILES: C[Si](C)(C)CCOCn1nc(-c2ccncc2)c(C(N)=O)c1N]

A mixture of 5-amino-3-(pyridin-4-yl)-1-((2-(trimethylsilyl) ethoxy)methyl)-1H-pyrazole-4-carbonitrile (4.5 g, 14.3 mmol) and KOH (2.7 g, 71.4 mmol) in EtOH/H₂O (50 mL/10 mL) was stirred at 100° C. overnight. The reaction mixture was concentrated in vacuo, water was added, and the resulting solid was filtered and dried in vacuo to give the title compound (3.2 g, 67%) as a yellow solid. ES/MS m/z=334 (M+H).

### Preparation 27

### 3-(Pyridin-4-yl)-1-((2-(trimethylsilyl) ethoxy)methyl)-1,5-dihydro-4H-pyrazolo[3,4-d]pyrimidin-4-one

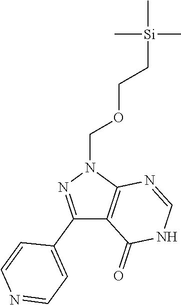 [SMILES: C[Si](C)(C)CCOCn1nc(-c2ccncc2)c2c(=O)[nH]cnc21]

A mixture of 5-amino-3-(pyridin-4-yl)-1-((2-(trimethylsilyl) ethoxy)methyl)-1H-pyrazole-4-carboxamide (2 g, 6 mmol) and formaldehyde (180 mg, 12 mmol) in toluene (20 mL) was stirred at 130° C. overnight. The reaction mixture was concentrated in vacuo to give the title compound (2.1 g, 99+%) as an off-white solid. ES/MS m/z=344 (M+H).

### Preparation 28

### 3-(Pyridin-4-yl)-1-((2-(trimethylsilyl) ethoxy)methyl)-1,5,6,7-tetrahydro-4H-pyrazolo[3,4-d]pyrimidin-4-one

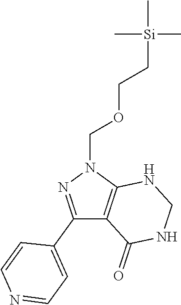 [SMILES: C[Si](C)(C)CCOCn1nc(-c2ccncc2)c2c1NCNC2=O]

To a mixture of 3-(pyridin-4-yl)-1-((2-(trimethylsilyl) ethoxy)methyl)-1,5-dihydro-4H-pyrazolo[3,4-d]pyrimidin-4-one (2.1 g, 6 mmol) in EtOH (20 mL) was added NaBH₄ (1.14 g, 30 mmol) which was stirred at ambient temperature for 0.5 h. The mixture was concentrated in vacuo. The residue was purified via silica gel flash chromatography eluting with 13% MeOH in DCM to give the title compound (700 mg, 34%) as an off-white solid. ES/MS m/z=346 (M+H).

### Preparation 29

### 3-Amino-1-(4-methoxybenzyl)-N-methyl-5-(pyridin-4-yl)-1H-pyrazole-4-carboxamide

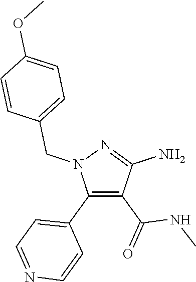 [SMILES: CNC(=O)c1c(N)nn(Cc2ccc(OC)cc2)c1-c1ccncc1]

A mixture of 3-amino-1-[(4-methoxyphenyl)methyl]-5-(4-pyridyl) pyrazole-4-carboxylic acid (1.2 g, 3.7 mmol), methylamine hydrochloride (726 mg, 11 mmol), HATU (1.7 g, 4.4 mmol), and DIPEA (1.4 g, 11 mmol) in DMA (10 mL) was stirred at ambient temperature for 6 hrs. To the reaction mixture was added water (50 mL) which was then extracted with EtOAc (100 mL×2). The combined organic layers were dried and concentrated in vacuo to give the crude product, which was purified via silica gel flash chromatography eluting with 10% MeOH in DCM to give the title compound (250 mg, 20%) as a yellow solid. ES/MS m/z=338 (M+H).

### Preparation 30

### 3-(3-(4-Chlorophenyl)propanamido)-1-(4-methoxybenzyl)-N-methyl-5-(pyridin-4-yl)-1H-pyrazole-4-carboxamide

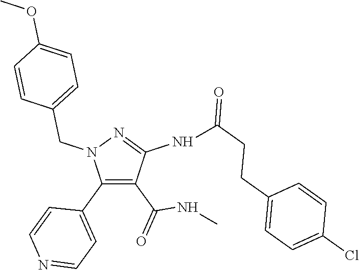 [SMILES: CNC(=O)c1c(NC(=O)CCc2ccc(Cl)cc2)nn(Cc2ccc(OC)cc2)c1-c1ccncc1]

A mixture of 3-amino-1-(4-methoxybenzyl)-N-methyl-5-(pyridin-4-yl)-1H-pyrazole-4-carboxamide (250 mg, 0.7 mmol), 3-(4-chlorophenyl)propanoic acid (205 mg, 1.1 mmol), T3P (700 mg, 2.2 mmol), and DIPEA (193 mg, 1.5 mmol) in DMA (6 mL) was stirred at ambient temperature for 3 hrs. To the reaction mixture was added water (10 mL) which was extracted with EtOAc (40 mL×2). The combined organic layers were dried over Na₂SO₄ and concentrated in vacuo to give crude product, which was purified via silica gel flash chromatography eluting with 2:1 petroleum ether:EtOAc to give the title compound (240 mg, 65%) as a white solid. ES/MS m/z=504 (M+H).

### EXAMPLE 1

### 3-(6-Chloropyridin-3-yl)-N-(4-oxo-4,5-dihydro-2H-pyrazolo[4,3-c][1,7]naphthyridin-3-yl)propanamide

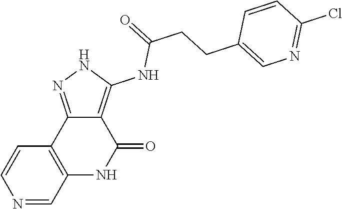 [SMILES: O=C(CCc1ccc(Cl)nc1)Nc1[nH]nc2c1c(=O)[nH]c1cnccc12]

Step 1: A mixture of 3-amino-1-(4-methoxybenzyl)-1,5-dihydro-4H-pyrazolo[4,3-c][1,7]naphthyridin-4-one (1.6 g, 5.0 mmol), 3-(6-chloropyridin-3-yl)propanoic acid (3.7 g, 20 mmol), T3P (6.4 g, 20 mmol), and DIPEA (2.6 g, 20 mmol) in DMA (20 mL) was stirred at 85° C. for 16 hrs. Water (30 mL) was added to the reaction mixture and the mixture extracted with EtOAc (100 mL×2). The combined organic layers were dried over Na₂SO₄ and concentrated in vacuo to give the 3-(6-chloropyridin-2-yl)-N-(5-(3-(6-chloropyridin-2-yl)propanoyl)-1-(4-methoxybenzyl)-4-oxo-4,5-dihydro-1H-pyrazolo[4,3-c][1,7]naphthyridin-3-yl)propanamide (1.5 g, 45%) as a yellow solid. ES/MS m/z=656 (M+H).

Step 2: A mixture of 3-(6-chloropyridin-2-yl)-N-(5-(3-(6-chloropyridin-2-yl)propanoyl)-1-(4-methoxybenzyl)-4-oxo-4,5-dihydro-1H-pyrazolo[4,3-c][1,7]naphthyridin-3-yl)propanamide (1.5 g, 2.3 mmol) and K₂CO₃ (345 mg, 2.5 mmol) in MeOH (20 mL) was stirred at 60° C. for 2 hrs. To the reaction mixture was added water (40 mL) and EtOAc (80 mL). The resulting solid was filtered, and the filter cake dried in vacuo to give 3-(6-chloropyridin-2-yl)-N-(1-(4-methoxybenzyl)-4-oxo-4,5-dihydro-1H-pyrazolo[4,3-c][1,7]naphthyridin-3-yl)propanamide (1.0 g, 91%) as a yellow solid. ES/MS m/z=489 (M+H).

Step 3: TFA (10 mL) was added to a mixture of 3-(6-chloropyridin-2-yl)-N-(1-(4-methoxybenzyl)-4-oxo-4,5-dihydro-1H-pyrazolo[4,3-c][1,7]naphthyridin-3-yl)propanamide (1.0 g, 2.0 mmol) in DCM (10 mL) and the reaction mixture stirred at ambient temperature for 4 hrs. The reaction mixture was concentrated in vacuo to give a crude product. To the crude product was added water (2 mL) and saturated aqueous NaHCO₃ (8 mL). The resulting precipitate was filtered and dried in vacuo to give the title compound (668 mg, 88%) as a white solid. ES/MS m/z=369 (M+H).

The compounds in Table 6 were prepared in a manner essentially analogous to that found in Example 1.

TABLE 6

| Ex. | | | |
|---|---|---|---|
| No | Chemical Name | Structure | ES/MS m/z |
| 2 | 3-(6-Fluoropyridin-3-y1)-N-(4- oxo-4,5-dihydro-2H- pyrazolo[4,3- c][1,7]naphthyridin-3- yl)propanamide | 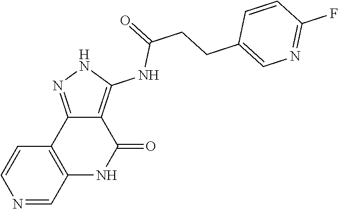 [SMILES: O=C(CCc1ccc(F)nc1)Nc1[nH]nc2c1c(=O)[nH]c1cnccc12] | 353 (M + H) |
| 3 | 3-(4-Chlorophenyl)-N-(4-oxo- 4,5-dihydro-2H-pyrazolo[4,3- c][1,7]naphthyridin-3- yl)propanamide | 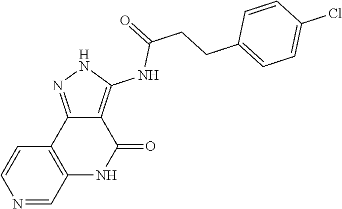 [SMILES: O=C(CCc1ccc(Cl)cc1)Nc1[nH]nc2c1c(=O)[nH]c1cnccc12] | 368 (M + H) |
| 4 | 3-(4-Chloro-3-fluorophenyl)- N-(4-oxo-4,5-dihydro-2H- pyrazolo[4,3- c][1,7]naphthyridin-3- yl)propanamide | 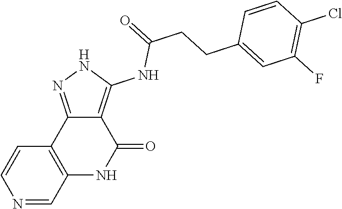 [SMILES: O=C(CCc1ccc(Cl)c(F)c1)Nc1[nH]nc2c1c(=O)[nH]c1cnccc12] | 386 (M + H) |
| 5 | 3-(6-Chloro-5-fluoro-3- pyridyl)-N-(4-oxo-2,5- dihydropyrazolo[4,3- c][1,7]naphthyridin-3- y1)propanamide | 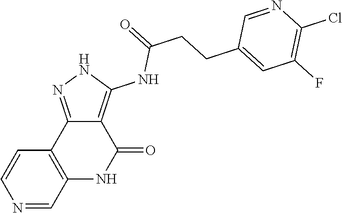 [SMILES: O=C(CCc1cnc(Cl)c(F)c1)Nc1[nH]nc2c1c(=O)[nH]c1cnccc12] | 387 (M + H) |
| 6 | 3-(3,4-Difluorophenyl)-N-(4- oxo-4,5-dihydro-2H- pyrazolo[4,3- c][1,7]naphthyridin-3- yl)propanamide | 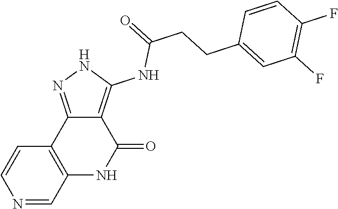 [SMILES: O=C(CCc1ccc(F)c(F)c1)Nc1[nH]nc2c1c(=O)[nH]c1cnccc12] | 370 (M + H) |

### EXAMPLE 7

### 5-(3-(3,4-Dichlorophenyl)propanamido)-3-(pyridin-4-yl)-1H-pyrazole-4-carboxamide

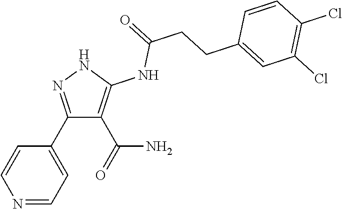 [SMILES: NC(=O)c1c(-c2ccncc2)n[nH]c1NC(=O)CCc1ccc(Cl)c(Cl)c1]

Step 1: A mixture of 3-amino-1-(4-methoxybenzyl)-5-(pyridin-4-yl)-1H-pyrazole-4-carboxamide (200 mg, 0.6 mmol), 3-(3,4-dichlorophenyl)propanoic acid (263 mg, 1.2 mmol), HATU (456 mg, 1.2 mmol), and DIPEA (232 mg, 1.8 mmol) in DMA (4 mL) was stirred at 65° C. for 3 hrs. To the reaction mixture was added water (10 mL) which was extracted with EtOAc (30 mL×2). The combined organic layers were dried over Na₂SO₄ and concentrated in vacuo to give 3-(3-(3,4-dichlorophenyl)propanamido)-1-(4-methoxybenzyl)-5-(pyridin-4-yl)-1H-pyrazole-4-carboxamide (200 mg, 62%) as a yellow solid. ES/MS m/z=524 (M+H).

Step 2: To a mixture of 3-(3-(3,4-dichlorophenyl)propanamido)-1-(4-methoxybenzyl)-5-(pyridin-4-yl)-1H-pyrazole-4-carboxamide (200 mg, 0.38 mmol) in DCM (3 mL) was added TFA (3 mL), and the reaction was stirred at ambient temperature for 4 hrs. The reaction mixture was concentrated in vacuo. To the residue was added water (2 mL) and saturated aqueous NaHCO₃ (10 mL). The resulting precipitate was filtered and dried in vacuo to give the title compound (137 mg, 88%) as a white solid. ES/MS m/z=404 (M+H).

The compounds in Table 7 were prepared in a manner essentially analogous to that found in Example 7.

TABLE 7

| Ex | | | |
|---|---|---|---|
| No | Chemical Name | Structure | ES/MS m/z |
| 8 | 5-(3-(3,4- Difluorophenyl)propanamido)- 3-(pyridin-4-y1)-1H- pyrazole-4-carboxamide | 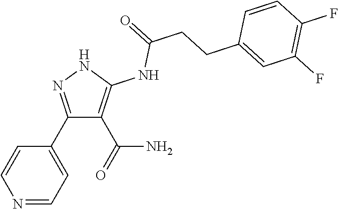 [SMILES: NC(=O)c1c(-c2ccncc2)n[nH]c1NC(=O)CCc1ccc(F)c(F)c1] | 372 (M + H) |
| 9 | 5-(3-(6-Chloropyridin-3- yl)propanamido)-3-(pyridin- 4-y1)-1H-pyrazole-4- carboxamide | 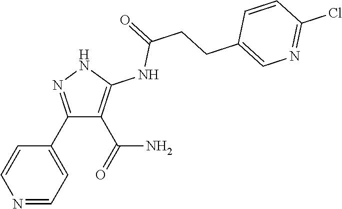 [SMILES: NC(=O)c1c(-c2ccncc2)n[nH]c1NC(=O)CCc1ccc(Cl)nc1] | 371 (M + H) |
| 10 | 5-(3-(4- Fluorophenyl)propanamido)- 3-(pyridin-4-yl)-1H-pyrazole- 4-carboxamide | 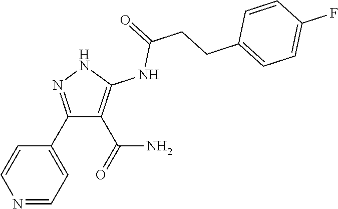 [SMILES: NC(=O)c1c(-c2ccncc2)n[nH]c1NC(=O)CCc1ccc(F)cc1] | 354 (M + H) |
| 11 | 5-(3-(3,5- Difluorophenyl)propanamido)- 3-(pyridin-4-y1)-1H- pyrazole-4-carboxamide- | 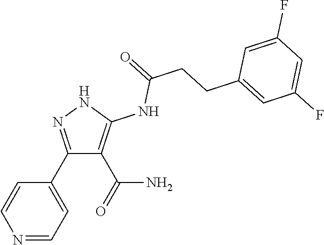 [SMILES: NC(=O)c1c(-c2ccncc2)n[nH]c1NC(=O)CCc1cc(F)cc(F)c1] | 372 (M + H) |
| 12 | 5-(3-(6-Chloro-5- fluoropyridin-3- yl)propanamido)-3-(pyridin- 4-yl)-1H-pyrazole-4- carboxamide | 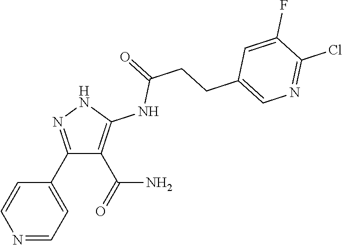 [SMILES: NC(=O)c1c(-c2ccncc2)n[nH]c1NC(=O)CCc1cnc(Cl)c(F)c1] | 389 (M + H) |
| 13 | 5-(3-(4- Chlorophenyl)propanamido)- 3-(pyridin-4-yl)-1H-pyrazole- 4-carboxamide | 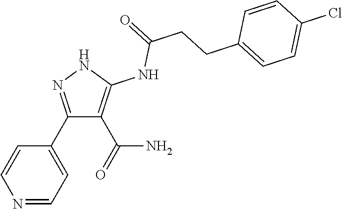 [SMILES: NC(=O)c1c(-c2ccncc2)n[nH]c1NC(=O)CCc1ccc(Cl)cc1] | 370 (M + H) |
| 14 | 5-(3-(4-Chloro-3- fluorophenyl)propanamido)- 3-(pyridin-4-yl)-1H-pyrazole- 4-carboxamide | 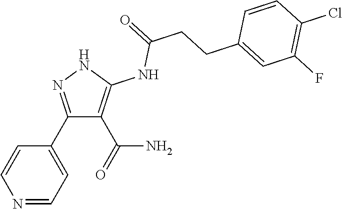 [SMILES: NC(=O)c1c(-c2ccncc2)n[nH]c1NC(=O)CCc1ccc(Cl)c(F)c1] | 388 (M + H) |
| 15 | 5-(3-(5-Chlorothiophen-2- yl)propanamido)-3 -(pyridin- 4-y1)-1H-pyrazole-4- carboxamide | 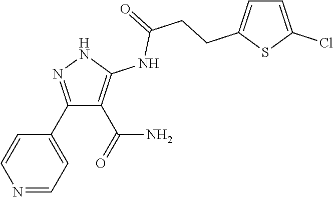 [SMILES: NC(=O)c1c(-c2ccncc2)n[nH]c1NC(=O)CCc1ccc(Cl)s1] | 376 (M + H) |
| 16 | 5-(3-(6-Fluoropyridin-3- yl)propanamido)-3-(5- methylpyridazin-4-yl)-1H- pyrazole-4-carboxamide | 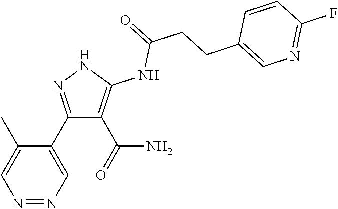 [SMILES: Cc1cnncc1-c1n[nH]c(NC(=O)CCc2ccc(F)nc2)c1C(N)=O] | 370 (M + H) |
| 17 | 5-(3-(4-Chloro-3- fluorophenyl)propanamido)- 3-(5-methylpyridazin-4-y1)- 1H-pyrazole-4-carboxamide | 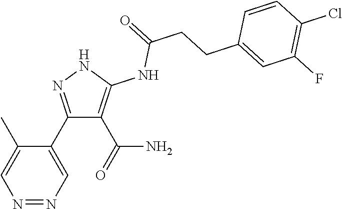 [SMILES: Cc1cnncc1-c1n[nH]c(NC(=O)CCc2ccc(Cl)c(F)c2)c1C(N)=O] | 403 (M + H) |
| 18 | 5-(3-(6-Fluoropyridin-3- yl)propanamido)-N-methyl-3- (pyridazin-4-y1)-1H-pyrazole- 4-carboxamide | 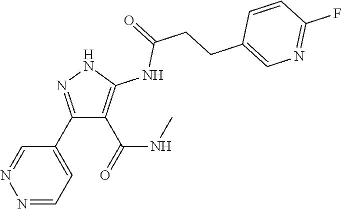 [SMILES: CNC(=O)c1c(-c2ccnnc2)n[nH]c1NC(=O)CCc1ccc(F)nc1] | 371 (M + H) |
| 19 | 5-(3-(6-Fluoropyridin-3- yl)propanamido)-3- (pyridazin-4-yl)-1H-pyrazole- 4-carboxamide | 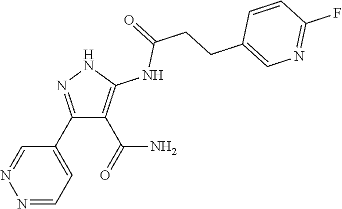 [SMILES: NC(=O)c1c(-c2ccnnc2)n[nH]c1NC(=O)CCc1ccc(F)nc1] | 357 (M + H) |
| 20 | 5-(3-(6-Chloro-5- fluoropyridin-3- yl)propanamido)-3- (pyridazin-4-y1)-1H-pyrazole- 4-carboxamide | 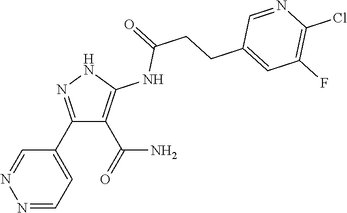 [SMILES: NC(=O)c1c(-c2ccnnc2)n[nH]c1NC(=O)CCc1cnc(Cl)c(F)c1] | 391 (M + H) |
| 21 | 5-(3-(4-Chloro-3- fluorophenyl)propanamido)- 3-(pyridazin-4-yl)-1H- pyrazole-4-carboxamide | 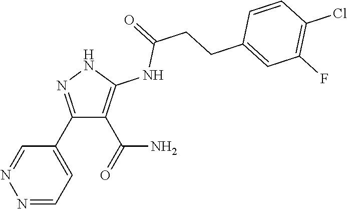 [SMILES: NC(=O)c1c(-c2ccnnc2)n[nH]c1NC(=O)CCc1ccc(Cl)c(F)c1] | 390 (M + H) |

### EXAMPLE 22

### 5-(3-(6-Fluoropyridin-3-yl)propanamido)-3-(pyridin-4-yl)-1H-pyrazole-4-carboxamide

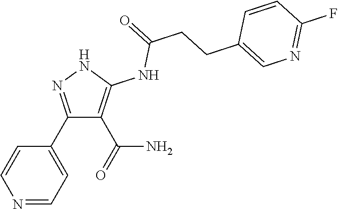 [SMILES: NC(=O)c1c(-c2ccncc2)n[nH]c1NC(=O)CCc1ccc(F)nc1]

Step 1: A mixture of 3-amino-1-(4-methoxybenzyl)-5-(pyridin-4-yl)-1H-pyrazole-4-carboxylic acid (4.0 g, 12.3 mmol), (2,4-dimethoxyphenyl) methanamine (3.4 g, 18.5 mmol), HATU (5.6 g, 14.8 mmol), and DIPEA (3.2 g, 24.6 mmol) in DMF (50 mL) was stirred at ambient temperature for 3 hrs. To the reaction mixture was added water (50 mL) which was extracted with EtOAc (100 mL×2). The combined organic layers were dried over Na₂SO₄ and concentrated in vacuo. The residue was purified via silica gel flash chromatography eluting with 10% MeOH in DCM to give 3-amino-N-(2,4-dimethoxybenzyl)-1-(4-methoxybenzyl)-5-(pyridin-4-yl)-1H-pyrazole-4-carboxamide (2.0 g, 35%) as a yellow solid. ES/MS m/z=474 (M+H).

Step 2: A mixture of 3-amino-N-(2,4-dimethoxybenzyl)-1-(4-methoxybenzyl)-5-(pyridin-4-yl)-1H-pyrazole-4-carboxamide (100 mg, 0.2 mmol), 3-(6-fluoropyridin-3-yl)propanoic acid (68 mg, 0.4 mmol), T3P (382 mg, 0.6 mmol), and DIPEA (52 mg, 0.4 mmol) in DMA (3 mL) was stirred at ambient temperature for 3 hrs. The reaction mixture was concentrated in vacuo. The resulting residue was purified via silica gel flash chromatography eluting with 5% MeOH in DCM to give N-(2,4-dimethoxybenzyl)-3-(3-(6-fluoropyridin-2-yl)propanamido)-1-(4-methoxybenzyl)-5-(pyridin-4-yl)-1H-pyrazole-4-carboxamide (100 mg, 76%) as a white solid. ES/MS m/z=625 (M+H).

Step 3: To a mixture of N-(2,4-dimethoxybenzyl)-3-(3-(6-fluoropyridin-2-yl)propanamido)-1-(4-methoxybenzyl)-5-(pyridin-4-yl)-1H-pyrazole-4-carboxamide (100 mg, 0.15 mmol) in DCM (2 mL) was added TFA (1 mL) and TfOH (0.2 mL). The mixture was stirred at ambient temperature for 2 hrs. then concentrated in vacuo. To the residue was added water (10 mL) and saturated aqueous NaHCO₃ (50 mL), and the mixture was extracted with EtOAc (50 mL×2). The combined organic layers were dried over Na₂SO₄ and concentrated in vacuo. The resulting residue was purified by Prep-HPLC (Water:MeCN=2:3) to give the title compound (11 mg, 19%) as a white solid. ES/MS m/z=355 (M+H).

The compounds in Table 8 were prepared in a manner essentially analogous to that found in Example 22.

TABLE 8

| Ex | | | |
|---|---|---|---|
| No | Chemical Name | Structure | ES/MS m/z |
| 23 | 3-(Pyridin-4-y1)-5-(3-(4- (trifluoromethyl)phenyl) propanamido)-1H- pyrazole-4- carboxamide | 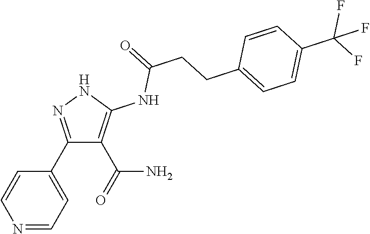 [SMILES: NC(=O)c1c(-c2ccncc2)n[nH]c1NC(=O)CCc1ccc(C(F)(F)F)cc1] | 404 (M + H) |

### EXAMPLE 24

### 3-(3-(4-chlorophenyl)propanamido)-N-methyl-5-(pyridin-4-yl)-1H-pyrazole-4-carboxamide

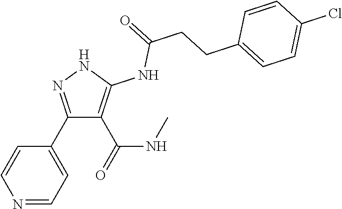 [SMILES: CNC(=O)c1c(-c2ccncc2)n[nH]c1NC(=O)CCc1ccc(Cl)cc1]

To a mixture of 3-(3-(4-chlorophenyl)propanamido)-1-(4-methoxybenzyl)-N-methyl-5-(pyridin-4-yl)-1H-pyrazole-4-carboxamide (240 mg, 0.5 mmol) in DCM (2 mL) was added TFA (2 mL). The mixture was stirred at ambient temperature for 2 hrs. then concentrated in vacuo. To the resulting residue was added water (4 mL) and saturated aqueous NaHCO₃ (16 mL) which was extracted with EtOAc (40 mL×2). The combined organic layers were dried over Na₂SO₄ and concentrated in vacuo. The residue was purified via silica gel flash chromatography eluting with 5:1 DCM:MeOH to give the title compound (80 mg, 44%) as a white solid. ES/MS m/z=384 (M+H).

### EXAMPLE 25

### 7-(3-(4-Chlorophenyl)propanoyl)-3-(pyridin-4-yl)-1,5,6,7-tetrahydro-4H-pyrazolo[3,4-d]pyrimidin-4-one

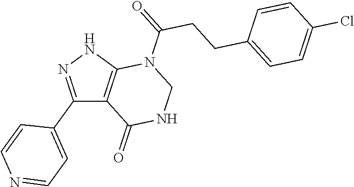 [SMILES: O=C1NCN(C(=O)CCc2ccc(Cl)cc2)c2[nH]nc(-c3ccncc3)c21]

Step 1: A mixture of 3-(pyridin-4-yl)-1-((2-(trimethylsilyl) ethoxy)methyl)-1,5,6,7-tetrahydro-4H-pyrazolo[3,4-d]pyrimidin-4-one (150 mg, 0.4 mmol), 3-(4-chlorophenyl)propanoic acid (93 mg, 0.5 mmol), T3P (226 mg, 0.7 mmol), and DIPEA (0.2 mL) in DMA (5 mL) was stirred at 100° C. overnight. To the mixture was added saturated aqueous NaHCO₃ solution (30 mL), which was then extracted with EtOAc (30 mL×3). The combined organic layers were dried over Na₂SO₄ and concentrated in vacuo. The residue was purified via silica gel flash chromatography eluting with 3% MeOH in DCM to give 7-(3-(4-chlorophenyl)propanoyl)-3-(pyridin-4-yl)-1-((2-(trimethylsilyl) ethoxy)methyl)-1,5,6,7-tetrahydro-4H-pyrazolo[3,4-d]pyrimidin-4-one (140 mg, 63%) as a yellow solid. ES/MS m/z=512 (M+H).

Step 2: A mixture of 7-(3-(4-chlorophenyl)propanoyl)-3-(pyridin-4-yl)-1-((2-(trimethylsilyl) ethoxy)methyl)-1,5,6,7-tetrahydro-4H-pyrazolo[3,4-d]pyrimidin-4-one (140 mg, 0.27 mmol), TFA (1 mL), and DCM (5 mL) was stirred at ambient temperature for 2 hrs. The mixture was concentrated in vacuo. The residue was purified by prep-HPLC (C18, MeCN in 10 mMol NH₄HCO₃; 22-52% in 9 min, rt=10.8 min) to give the title compound (4.4 mg, 4.2%) as a white solid. ES/MS m/z=382 (M+H).

The examples in Table 9 were prepared essentially the same as described for Example 25 using the appropriate starting materials and reagents.

TABLE 9

| | | | ES/MS |
|---|---|---|---|
| Example | Chemical Name | Structure | m/z |
| 26 | 7-(3-(4-Chloro-3- fluorophenyl)propano y1)-3-(pyridin-4-y1)- 1,5,6,7-tetrahydro-4H- pyrazolo[3,4- d]pyrimidin-4-one | 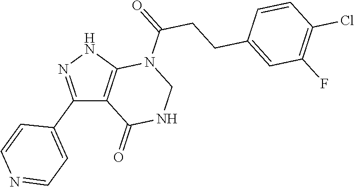 [SMILES: O=C1NCN(C(=O)CCc2ccc(Cl)c(F)c2)c2[nH]nc(-c3ccncc3)c21] | 400 (M + H) |
| 27 | 7-(3-(6-Chloro-5- fluoropyridin-3- y1)propanoyl)-3- (pyridin-4-y1)-1,5,6,7- tetrahydro-4H- pyrazolo[3,4- d]pyrimidin-4-one | 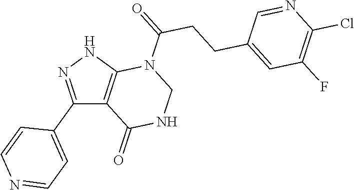 [SMILES: O=C1NCN(C(=O)CCc2cnc(Cl)c(F)c2)c2[nH]nc(-c3ccncc3)c21] | 401 (M + H) |
| 28 | 7-(3-(6-Chloropyridin- 3-yl)propanoyl)-3- (pyridin-4-yl)-1,5,6,7- tetrahydro-4H- pyrazolo[3,4- d]pyrimidin-4-one | 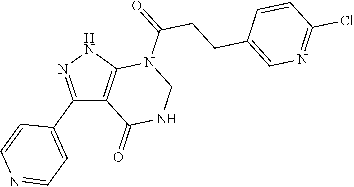 [SMILES: O=C1NCN(C(=O)CCc2ccc(Cl)nc2)c2[nH]nc(-c3ccncc3)c21] | 383 (M + H) |

### ARM-SAM-TIR SARM1 IC50 Assay

This describes an assay of ARM-SAM-TIR NADase activity and use of this assay to measure the efficacy of compounds of the present invention to block SARM1 mediated NAD+ cleavage. This assay was optimized in such a way as to characterize the efficacy of the compounds of the present invention to inhibit SARM1 activity and to calculate an IC50 value for each compound. This assay makes use of full length SARM1, which encompasses the ARM, SAM and TIR domains. As demonstrated herein, expression of this fragment without the autoinhibitory N-terminal domain generates a constitutively active enzyme that cleaves NAD+.

### Preparation of ARM-SAM-TIR Lysate (STL)

NRK1-HEK293T cells were seeded onto 150 cm² plates at 20×106 cells per plate. The next day, the cells were transfected with 15 μg ARM-SAM-TIR expression plasmid (SEQ ID NO: 1 as disclosed in WO 2019/236879, Pages 77-81; Paragraph) [0310]).

The cultures were supplemented with 1 mM NR at time of transfection to minimize toxicity from ARM-SAM-TIR overexpression. Forty-eight hours after transfection, cells were harvested, pelleted by centrifugation at 1,000 rpm (Sorvall ST 16R centrifuge, Thermo Fisher), and washed once with cold PBS (0.01 M phosphate buffered saline NaCl 0.138 M; KCl 0.0027 M; pH 7.4). The cells were resuspended in PBS with protease inhibitors (Complete™ protease inhibitor cocktail, Roche product #11873580001) and cell lysates were prepared by sonication (Branson Sonifer 450, output=3, 20 episodes of stroke). The lysates were centrifuged (12,000×g for 10 min at 4° C.) to remove cell debris and the supernatants (containing ARM-SAM-TIR protein) were stored at −80° C. for later use in the in vitro ARM-SAM-TIR NADase assay (see below). Protein concentration was determined by the Bicinchoninic (BCA) method and used to normalize lysate concentrations.

### ARM-SAM-TIR IC50 Assay of Formula I Compounds.

The enzymatic assay was performed in a 384-well polypropylene plate in Dulbecco's PBS buffer in a final assay volume of 20 μL. ARM-SAM-TIR lysate with a final concentration of 5 μg/mL was pre-incubated with the respective compound at 1% DMSO final assay concentration over 2 hrs. at room temperature. The reaction was initiated by addition of 5 μM final assay concentration of NAD+ as substrate. After a 2 hrs. ambient temperature incubation, the reaction was terminated with 40 μL of stop solution of 7.5% trichloroacetic acid in acetonitrile. The NAD+ and ADPR concentrations were analyzed by a RapidFire High Throughput Mass Spectrometry System (Agilent Technologies, Santa Clara, CA) using an API4000 triple quadrupole mass spectrometer (AB Sciex Framingham, MA).

Results are presented below in Table 10. Compounds having an activity designated as “A” provided an IC₅₀<50 nM; compounds having an activity designated as “B” provided an IC₅₀ 51-100 nM; compounds having an activity designated as “C” provided an IC₅₀ 101-500 nM; compounds having an activity designated as “D” provided an IC₅₀ 501-1000 nM; compounds having an activity designated as “E” provided an IC₅₀>1000 nM.

TABLE 10
hSARM1 IC₅₀ Assay

| Example No. | IC₅₀ (nM) |
|---|---|
| 1 | C |
| 2 | C |
| 3 | A |
| 4 | A |
| 5 | B |
| 6 | A |
| 7 | B |
| 8 | C |
| 9 | C |
| 10 | C |
| 11 | D |
| 12 | C |
| 13 | A |
| 14 | A |
| 15 | C |
| 16 | E |
| 17 | D |
| 18 | E |
| 19 | E |
| 20 | C |
| 21 | B |
| 22 | D |
| 23 | C |
| 24 | C |
| 25 | C |
| 26 | C |
| 27 | E |
| 28 | E |

The results shown in Table 10 above demonstrate that the compounds of the present invention possess hSARM1 inhibitory activity.

1. A compound of the formula 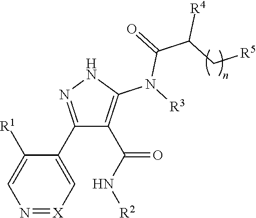
wherein
X is selected from CH and N;
R¹, R², R³ and R⁴ are each independently selected from H and C₁₋₄ alkyl, wherein R¹ and R² may optionally form a 6-membered heterocyclic ring together with the atoms they are attached to, wherein R² and R³ may optionally form a 6- to 7-membered heterocyclic ring together with the atoms they are attached to, and wherein R³ and R⁴ may optionally form a 5- to 6-membered heterocyclic ring together with the atoms they are attached to;
n is 0, 1 or 2;
R⁵ is selected from phenyl optionally substituted with 1 to 3 R^{m} and 5- to 6-membered heteroaryl containing 1 to 3 heteroatoms selected from oxygen, nitrogen and sulfur and optionally substituted with 1 to 3 R^{m}; and
R^{m} is selected from halogen, cyano, C₁₋₄ alkyl, trihalomethyl and —OC₁₋₄ alkyl, or a pharmaceutically acceptable salt thereof.

2. The compound according to claim 1 or a pharmaceutically acceptable salt thereof, wherein R¹, R², R³ and R⁴ are each independently selected from H and C₁₋₄ alkyl.

3. The compound according to claim 2 or a pharmaceutically acceptable salt thereof, wherein R¹, R², R³ and R⁴ are each independently selected from H, methyl, ethyl, n-propyl and iso-propyl.

4. The compound according to claim 1, which is 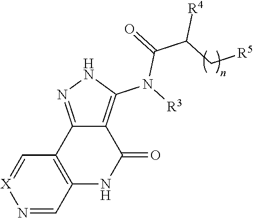
or a pharmaceutically acceptable salt thereof.

5. The compound according to claim 4 or a pharmaceutically acceptable salt thereof, wherein R³ and R⁴ are each independently selected from H, methyl, ethyl, n-propyl and iso-propyl.

6. The compound according to claim 1, which is 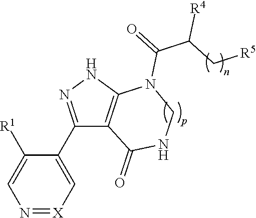
wherein
p is 1 or 2;
or a pharmaceutically acceptable salt thereof.

7. The compound according to claim 6 or a pharmaceutically acceptable salt thereof, wherein R¹ and R⁴ are each independently selected from H, methyl, ethyl, n-propyl and iso-propyl.

8. The compound according to claim 1, which is 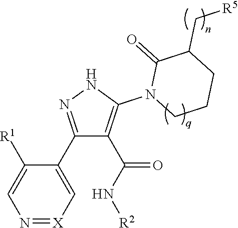
wherein
q is 0 or 1;
or a pharmaceutically acceptable salt thereof.

9. The compound according to claim 8 or a pharmaceutically acceptable salt thereof, wherein R¹ and R² are each independently selected from H, methyl, ethyl, n-propyl and iso-propyl.

10. The compound according to claim 1 or a pharmaceutically acceptable salt thereof, wherein X is CH.

11. The compound according to claim 1 or a pharmaceutically acceptable salt thereof, wherein X is N.

12. The compound according to claim 1 or a pharmaceutically acceptable salt thereof, wherein n is 1.

13. The compound according to claim 6 or a pharmaceutically acceptable salt thereof, wherein p is 1.

14. The compound according to claim 8 or a pharmaceutically acceptable salt thereof, wherein q is 0.

15. The compound according to claim 1 or a pharmaceutically acceptable salt thereof, wherein R⁵ is phenyl optionally substituted with 1 to 3 R^{m}.

16. The compound according to claim 1 or a pharmaceutically acceptable salt thereof, wherein R⁵ is 5- to 6-membered heteroaryl containing 1 to 3 heteroatoms selected from oxygen, nitrogen and sulfur optionally substituted with 1 to 3 R^{m}.

17. The compound according to claim 1 or a pharmaceutically acceptable salt thereof, wherein each R^{m} is independently selected from F, Cl, Br, I, cyano and trifluoromethyl.

18. The compound according to claim 1 or a pharmaceutically acceptable salt thereof, where in R⁵ is selected from the group consisting of:

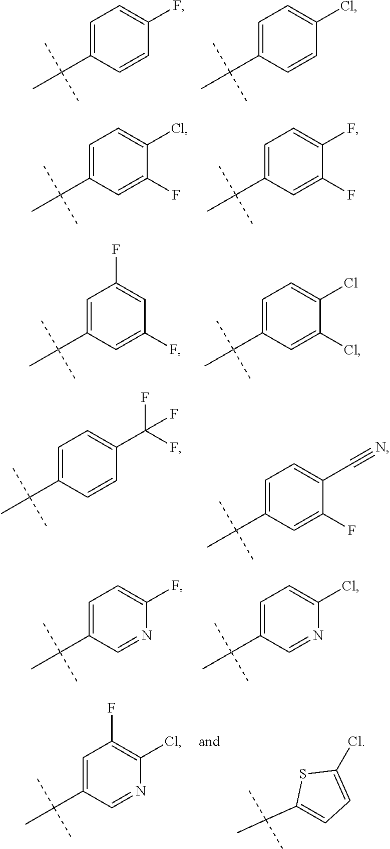

19. The compound according to claim 1, which is selected from: 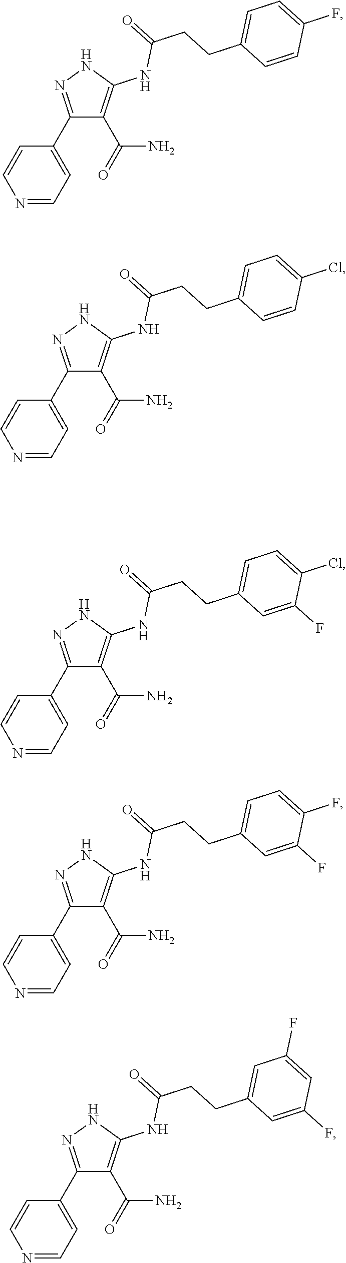 [SMILES: NC(=O)c1c(-c2ccncc2)n[nH]c1NC(=O)CCc1ccc(F)cc1 ; NC(=O)c1c(-c2ccncc2)n[nH]c1NC(=O)CCc1ccc(Cl)cc1 ; NC(=O)c1c(-c2ccncc2)n[nH]c1NC(=O)CCc1ccc(Cl)c(F)c1 ; NC(=O)c1c(-c2ccncc2)n[nH]c1NC(=O)CCc1ccc(F)c(F)c1 ; NC(=O)c1c(-c2ccncc2)n[nH]c1NC(=O)CCc1cc(F)cc(F)c1] 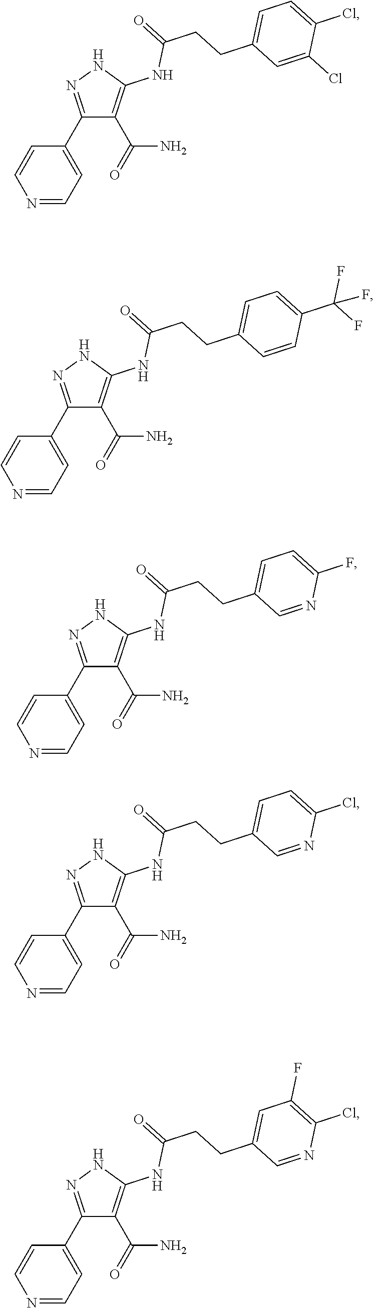 [SMILES: NC(=O)c1c(-c2ccncc2)n[nH]c1NC(=O)CCc1ccc(Cl)c(Cl)c1 ; NC(=O)c1c(-c2ccncc2)n[nH]c1NC(=O)CCc1ccc(C(F)(F)F)cc1 ; NC(=O)c1c(-c2ccncc2)n[nH]c1NC(=O)CCc1ccc(F)nc1 ; NC(=O)c1c(-c2ccncc2)n[nH]c1NC(=O)CCc1ccc(Cl)nc1 ; NC(=O)c1c(-c2ccncc2)n[nH]c1NC(=O)CCc1cnc(Cl)c(F)c1] 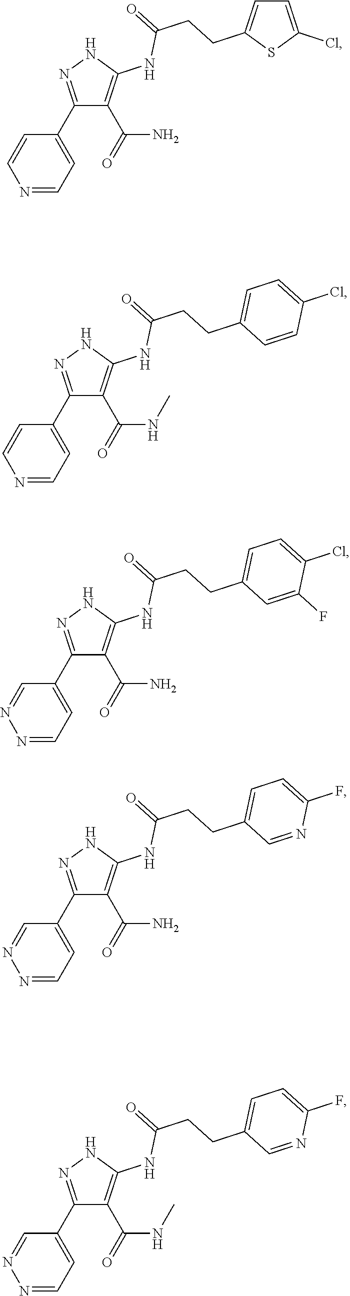 [SMILES: NC(=O)c1c(-c2ccncc2)n[nH]c1NC(=O)CCc1ccc(Cl)s1 ; CNC(=O)c1c(-c2ccncc2)n[nH]c1NC(=O)CCc1ccc(Cl)cc1 ; NC(=O)c1c(-c2ccnnc2)n[nH]c1NC(=O)CCc1ccc(Cl)c(F)c1 ; NC(=O)c1c(-c2ccnnc2)n[nH]c1NC(=O)CCc1ccc(F)nc1 ; CNC(=O)c1c(-c2ccnnc2)n[nH]c1NC(=O)CCc1ccc(F)nc1] 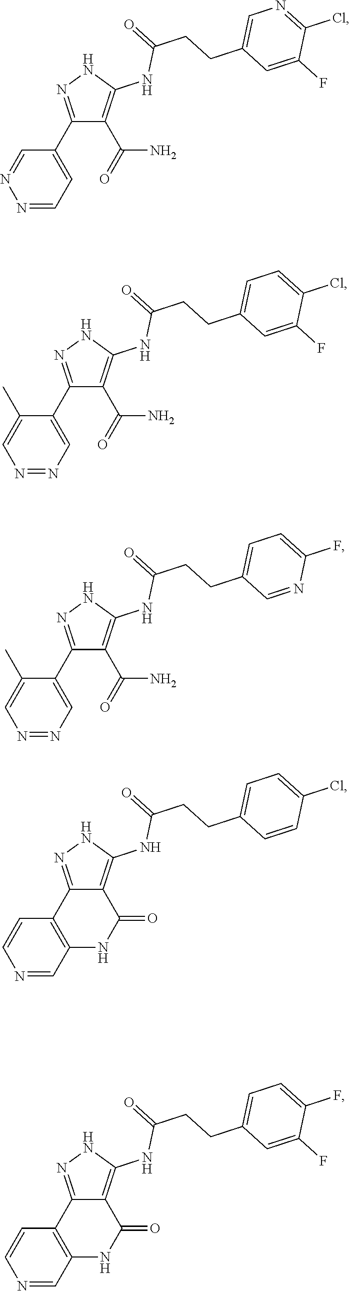 [SMILES: NC(=O)c1c(-c2ccnnc2)n[nH]c1NC(=O)CCc1cnc(Cl)c(F)c1 ; Cc1cnncc1-c1n[nH]c(NC(=O)CCc2ccc(Cl)c(F)c2)c1C(N)=O ; Cc1cnncc1-c1n[nH]c(NC(=O)CCc2ccc(F)nc2)c1C(N)=O ; O=C(CCc1ccc(Cl)cc1)Nc1[nH]nc2c1c(=O)[nH]c1cnccc12 ; O=C(CCc1ccc(F)c(F)c1)Nc1[nH]nc2c1c(=O)[nH]c1cnccc12] 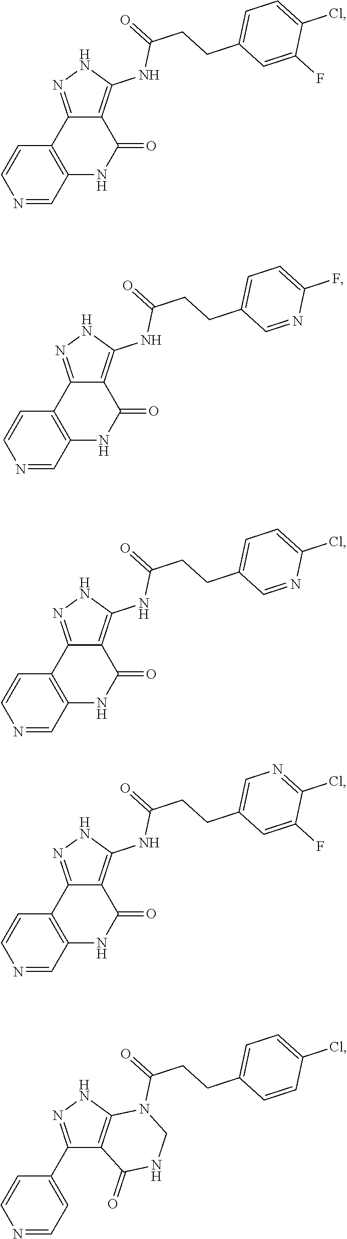 [SMILES: O=C(CCc1ccc(Cl)c(F)c1)Nc1[nH]nc2c1c(=O)[nH]c1cnccc12 ; O=C(CCc1ccc(F)nc1)Nc1[nH]nc2c1c(=O)[nH]c1cnccc12 ; O=C(CCc1ccc(Cl)nc1)Nc1[nH]nc2c1c(=O)[nH]c1cnccc12 ; O=C(CCc1cnc(Cl)c(F)c1)Nc1[nH]nc2c1c(=O)[nH]c1cnccc12 ; O=C1NCN(C(=O)CCc2ccc(Cl)cc2)c2[nH]nc(-c3ccncc3)c21] 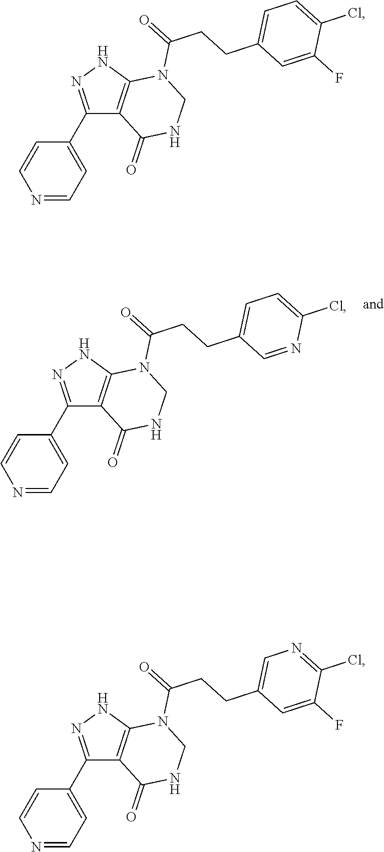 [SMILES: O=C1NCN(C(=O)CCc2ccc(Cl)c(F)c2)c2[nH]nc(-c3ccncc3)c21 ; O=C1NCN(C(=O)CCc2ccc(Cl)nc2)c2[nH]nc(-c3ccncc3)c21 ; O=C1NCN(C(=O)CCc2cnc(Cl)c(F)c2)c2[nH]nc(-c3ccncc3)c21]
or a pharmaceutically acceptable salt thereof.

20. A pharmaceutical composition comprising a compound according to claim 1 or a pharmaceutically acceptable salt thereof, with one or more pharmaceutically acceptable carriers, diluents, or excipients.

21. A method of treating or preventing a disease associated with axonal degeneration in a patient, comprising administering to a patient in need of such treatment an effective amount of a compound according to claim 1 or pharmaceutically acceptable salt thereof.

22. A method of treating or preventing a disease associated with SARM1 activation in a patient, comprising administering to a patient in need of such treatment an effective amount of a compound according to claim 1 or pharmaceutically acceptable salt thereof.

23. A method of treating or preventing a disease selected from amyotrophic lateral sclerosis (ALS), multiple sclerosis (MS), diabetic neuropathy and chemotherapy-induced peripheral neuropathy in a patient, comprising administering to a patient in need of such treatment an effective amount of a compound thereof according to claim 1 or pharmaceutically acceptable salt thereof.

24. (canceled)

25. (canceled)

26. (canceled)
